(19) United States
(12) Patent Application Publication
(10) Pub. No.: US 2006/0212090 A1
(43) Pub. Date: Sep. 21, 2006

(54) Method of treating cognitive disorders using neuromodulation

(75) Inventors: Andres M. Lozano, Toronto (CA); Helen S. Mayberg, Toronto (CA)

Correspondence Address: FULBRIGHT & JAWORSKI, LLP, 1301 MCKINNEY, SUITE 5100, HOUSTON, TX, 77010-3095, US

(73) Assignee: Functional Neuroscience Inc., Toronto (CA)

(21) Appl. No.: 11/364,977

(22) Filed: Mar. 1, 2006

Related U.S. Application Data
(60) Provisional application No. 60/657,462, filed on Mar. 1, 2005.

(51) Int. Cl. A61N 1/18

(52) U.S. Cl. 607/45

(57) ABSTRACT

The present invention involves a method and a system for using electrical stimulation and/or chemical stimulation to treat a cognitive impairment and/or disorder. More particularly, the method comprises surgically implanting an electrode and/or catheter that is in communication with a predetermined site which is coupled to a pulse generating source and/or infusion pump that release either an electrical signal and/or a pharmaceutical resulting in stimulation of the predetermined site thereby treating the cognitive disorder and/or enhancing the cognitive ability.

The present invention claims priority to U.S. Provisional Patent Application Ser. No. 60/657,462, filed Mar. 1, 2005, which is incorporated by reference herein in its entirety.

### TECHNICAL FIELD

This invention relates to nervous tissue stimulation for treating cognitive disorders and more particularly to modulating nervous tissue at a predetermined stimulation site in brain tissue.

### BACKGROUND OF THE INVENTION

Various disorders and diseases exist which affect cognition. Cognition can be generally described as including at least three different components: attention, learning, and memory. Each of these components and their respective levels affect the overall level of a subject's cognitive ability. For instance, while Alzheimer's Disease patients suffer from a loss of overall cognition and thus deterioration of each of these characteristics, it is the loss of memory that is most often associated with the disease. In other diseases patients suffer from cognitive impairment that is more predominately associated with different characteristics of cognition, for instance Attention Deficit Hyperactivity Disorder (ADHD), focuses on the individual's ability to maintain an attentive state. Other conditions include general dementias associated with other neurological diseases, aging, and treatment of conditions that can cause deleterious effects on mental capacity, such as cancer treatments, stroke/ischemia, and mental retardation. The present invention is directed toward the treatment of these and other similar disorders through the repair or amelioration of the cognitive deficits or impairments.

Cognition disorders create a variety of problems for today's society. Therefore, scientists have made efforts to develop cognitive enhancers or cognition activators. The cognition enhancers or activators that have been developed are generally classified to include nootropics, vasodilators, metabolic enhancers, psychostimulants, cholinergic agents, biogenic amines drugs, and neuropeptides. Vasodilators and metabolic enhancers (e.g. dihydroergotoxine) are mainly effective in the cognition disorders induced by cerebral vessel ligation-ischemia; however, they are ineffective in clinical use and with other types of cognition disorders. Of the developed cognition enhancers, typically only metabolic drugs are employed for clinical use, as others are still in the investigation stage. Of the nootropics for instance, piracetam activates the peripheral endocrine system, which is not appropriate for Alzheimer's Disease due to the high concentration of steroids produced in patients while tacrine, a cholinergic agent, has a variety of side effects including vomiting, diarrhea, and hepatotoxicity.

Ways to improve the cognitive abilities of diseased individuals have been the subject of various studies. Recently the cognitive state related to Alzheimer's Disease and different ways to improve patient's memory have been the subject of various approaches and strategies. In the case of Alzheimer's Disease, efforts to improve cognition, typically through the cholinergic pathways or though other brain transmitter pathways, have been investigated. This approach relies on the inhibition of acetyl cholinesterase enzymes through drug therapy. Acetyl cholinesterase is a major brain enzyme and manipulating its levels can result in various changes to other neurological functions and cause side effects. Cholinesterase inhibitors only produce some symptomatic improvement for a short time. Additionally, the use of cholinergic inhibitors only produces an improvement in a fraction of the Alzheimer's Disease patients with mid to moderate symptoms and is thus only a useful treatment for a small portion of the overall patient population. As a result, use of the cholinergic pathway for treatment of cognitive impairment, particularly in Alzheimer's Disease, has proven to be inadequate. Additionally, current treatments for cognitive improvement are limited to specific neurodegenerative diseases and have not proven effective in treatment across a broad range of cognitive conditions.

The use of electrical stimulation for treating neurological disease, including such disorders as movement disorders including Parkinson's disease, essential tremor, dystonia, and chronic pain, has been widely discussed in the literature. It has been recognized that electrical stimulation holds significant advantages over lesioning since lesioning destroys the nervous system tissue. In many instances, the preferred effect is to modulate neuronal activity. Electrical stimulation permits such modulation of the target neural structures and, equally importantly, does not require the destruction of nervous tissue. Such electrical stimulation procedures include electroconvulsive therapy (ECT), repetitive transcranial (rTMS) magnetic stimulation and vagal nerve stimulation (VNS).

Deep brain stimulation (DBS) has been applied to the treatment of central pain syndromes and movement disorders, and it is currently being explored as a therapy for epilepsy. For instance, U.S. Pat. No. 6, 016,449 and U.S. Pat. No. 6,176,242 disclose a system for the electrical stimulation of areas in the brain for the treatment of certain neurological diseases such as epilepsy, migraine headaches and Parkinson's disease.

Various electrical stimulation and/or drug infusion devices have been proposed for treating neurological disorders. Some devices stimulate through the skin, such as electrodes placed on the scalp. Other devices require significant surgical procedures for placement of electrodes, catheters, leads, and/or processing units. These devices may also require an external apparatus that needs to be strapped or otherwise affixed to the skin.

There still exists a need for the development of methods for the treatment for improved overall cognition, either through a specific characteristic of cognitive ability or general cognition. There also still exists a need for the development of methods for the improvement of cognitive enhancement whether or not it is related to a specific disease state or cognitive disorder. The methods and compositions of the present invention are needed and will greatly improve the clinical treatment for diminished cognitive ability whether related to a specific neurodegenerative disease, hypoxia, stroke or similar disorder. The methods and compositions also provide treatment and/or enhancement of the cognitive state.

### BRIEF SUMMARY OF THE INVENTION

The present invention relates to electrical and/or chemical stimulation applied to areas of the brain not considered in the prior art to play a role in enhancing cognition and/or alleviating or treating cognitive impairments or disorders and/or enhancing memory. In certain embodiments, the invention uses electrical stimulation and/or chemical stimulation (i.e., one or more pharmaceuticals) to treat cognitive impairments or enhance cognition. In addition to electrical and/or chemical stimulation, magnetic stimulation can also be used, such as transcranial magnetic stimulation (“TMS”). According to one embodiment of the invention, the stimulation modulates areas of the brain that exhibit altered activity in patients relative to neurologically and/or psychiatrically normal control subjects, thereby treating or preventing cognitive impairments or disorders. Such stimulation is likely to be produced by electrical stimulation, an excitatory neurotransmitter agonist(s), an inhibitory neurotransmitter antagonist(s), and/or a medication that increases the level of an excitatory neurotransmitter.

In addition to electrical and/or chemical stimulation, magnetic stimulation, ultrasonic stimulation and/or thermal stimulation can also be used. Magnetic stimulation can be provided by internally implanted probes or by externally applied directed magnetic fields. Thermal stimulation can be provided by using implanted probes that are regulated to produce or emit heat and/or cold temperatures.

Alternatively, affective disorders can be treated by utilizing other known methods to alter the neuronal activity of the above mentioned predetermined sites. For example, lesioning and mechanical disruption can be used as described by U.S. Pat. Nos. 6,629,973, 3,653,385, which is incorporated herein by reference in its entirety.

One embodiment of the present invention utilizes neurosurgical intervention to modulate neuronal activity in patients suffering from cognitive impairments and/or disorders. Such interventions include, applying electrical stimulation, herein termed “deep brain stimulation” or DBS, as is currently practiced to treat a number of disorders like Parkinson's disease. Other stimulations can include chemical stimulation such as through the use of pharmaceutical or drug pumps, for example local delivery of neuroactive substances to disrupt or block the pathological activity stemming from or coursing through this area. It is envisioned that such stimulation (i.e., electrical, magnetic, chemical, thermal and/or ultrasonic) modulates the gray matter and white matter tracts in a predetermined area.

The predetermined site or target area can include but are limited to the subcallosal area, subgenual cingulate area, hypothalamus, orbital frontal cortex, anterior insula, medial frontal cortex, dorsolateral prefrontal, dorsal anterior cortex, posterior cingulate area, premotor, orbital frontal, parietal region, ventrolateral prefrontal, dorsal cingulate, dorsal anterior cingulate, caudate nucleus, anterior thalamus, nucleus accumbens; periaqueductal gray area, brainstem, and/or the surrounding or adjacent white matter tracts leading to or from the all of these listed areas or white matter tracts that are contiguous. Thus, stimulation of any of the above brain tissue areas, as well as any white matter tracts afferent to or efferent from the abovementioned brain tissue can result in alterations or changes that alleviate or improve the cognitive impairment and/or disorder of the subject. Still further, other stimulations may comprise magnetic stimulation and/or transplantation of cells.

In certain embodiments, the predetermined site is a subcallosal area. A subcallosal area includes, but is not limited to subgenual cingulate area, subcallosal gyrus area, ventral/medial prefrontal cortex area, ventral/medial white matter, Brodmann area 24, Brodmann area 25, and/or Brodmann area 10. More specifically, the predetermined site is a subgenual cingulate area, more preferably Brodmann area 25, Brodmann area 24 or Brodmann area 10.

Thus, the system and methods of the present invention have utility in treating clinical conditions and disorders in which impaired memory or a learning disorder occurs, either as a central feature or as an associated symptom. Examples of such conditions in which the system or method can be used to treat include Alzheimer's Disease, multi-infarct dementia and the Lewy-body variant of Alzheimer's Disease with or without association with Parkinson's Disease; Creutzfeld-Jakob Disease, Korsakow's disorder, attention deficit hyperactivity disorder, hypoxia, ischeamic stroke, anoxia, hypoglycemia, hyperglycemia, metabolic disorders, dystonia, chorea, tics and mycolonus, post-head injury, post-irradiation, mental retardation, general dementia, and “sundown” syndrome.

Still further, the system and method of the present invention can also be used to treat impaired memory or learning which is age-associated, is consequent upon electro-convulsive therapy or which is the result of brain damage caused, for example, by stroke, an anesthetic accident, head trauma, hypoglycemia, carbon monoxide poisoning, lithium intoxication or a vitamin deficiency.

Methods according to the invention are useful in the enhancement of cognition, prophylaxis and/or treatment of cognition disorders, wherein cognition disorders include, but are not limited to, disorders of learning acquisition, memory consolidation, and retrieval, as described herein. Yet further, the present invention can be used to improve motivation, attention, concentration and reward. Thus, the methods according to the present invention may be useful to treat attention deficit disorders, drug addiction, disorders of verbal fluency, aphasias, dysphasias, psychomotor retardation, and risk-taking behavior.

In further embodiments, the methods according to the present invention may be used to effect sleep, appetite, libido, neuroendrocine functions, memory and other disorders associated with these listed functions.

Certain embodiments of the present invention involve a method that comprises surgically implanting a device or stimulation system in communication with a predetermined site. The device or stimulation system is operated to stimulate the predetermined site thereby treating the cognitive impairment and/or enhancing cognitive abilites. The device or stimulation system may include a probe, for example, an electrode assembly (i.e., electrical stimulation lead), pharmaceutical-delivery assembly (i.e., catheters) or combinations of these (i.e., a catheter having at least one electrical stimulation lead) and/or a signal generator or signal source (i.e., electrical signal source, chemical signal source (i.e., pharmaceutical delivery pump) or magnetic signal source). The probe may be coupled to the electrical signal source, pharmaceutical delivery pump, or both which, in turn, is operated to stimulate the predetermined treatment site. Yet further, the probe and the signal generator or source can be incorporated together, wherein the signal generator and probe are formed into a unitary or single unit, such unit may comprise, one, two or more electrodes. These devices are known in the art as microstimulators, for example, Bion™ which is manufactured by Advanced Bionics Corporation.

Stimulation of the above mentioned predetermined areas includes stimulation of the gray matter and white matter tracts associated therewith that results in an alleviation or modulation of the cognitive impairment and/or disorder or results in cognitive enhancement. Associated white matter tracts includes the surrounding or adjacent white matter tracts leading to or from or white matter tracts that are contiguous with the area. Modulating the predetermined brain tissue area via electrical and/or chemical stimulation (i.e., pharmaceutical) and/or magnetic stimulation can result in increasing, decreasing, masking, altering, overriding or restoring neuronal activity resulting in treatment of the cognitive impairment and/or disorder or results in an increase or enhancement of cognition. Yet further, stimulation of a subcallosal area may result in modulation of neuronal activity of other areas of the brain, for example, Brodmann area 24, Brodmann area 25, Brodmann area 10, Brodmann area 9, the hypothalamus the brain stem, orbital frontal cortex (Brodmann area 32/Brodmann area 10), anterior insula, medial frontal cortex, dorsolateral prefrontal (Brodmann area 9/46), posterior cingulate area (Brodmann area 31), premotor (Brodmann area 6), parietal region (Brodmann area 40), ventrolateral prefrontal (Brodmann area 47), caudate nucleus, anterior thalamus, nucleus accumbens, frontal pole, periaqueductal gray area, and/or the surrounding or adjacent white matter tracts leading to or from the all of these listed areas or white matter tracts that are contiguous.

Another embodiment of the present invention comprises a method of treating the cognitive impairment and/or disorder comprising the steps of: surgically implanting an electrode in communication with a predetermined site; the electrode is coupled to or in communication with a pulse generation source; and an electrical signal is generated using the pulse generation source to modulate the predetermined site thereby treating the cognitive impairment and/or disorder.

In further embodiments, the method can comprise the steps of: surgically implanting a catheter having a proximal end coupled to a pump and a discharge portion for infusing a dosage of a pharmaceutical, wherein after implantation the discharge portion of the catheter is in communication with the predetermined stimulation site; and operating the pump to discharge the pharmaceutical through the discharge portion of the catheter into the stimulation site thereby treating the cognitive impairment and/or disorder. The pharmaceutical is selected from the group consisting of inhibitory neurotransmitter agonist, an excitatory neurotransmitter antagonist, an agent that increases the level of an inhibitory neurotransmitter, an agent that decrease the level of an excitatory neurotransmitter, and a local anesthetic agent. It is envisioned that chemical stimulation or pharmaceutical infusion can be preformed independently of electrical stimulation and/or in combination with electrical stimulation.

Another embodiment of the present invention is a method of treating a cognitive impairment and/or disorder comprising the steps of: surgically implanting an electrical stimulation lead having a proximal end and a stimulation portion, wherein after implantation the stimulation portion is in communication with a predetermined site; surgically implanting a catheter having a proximal end coupled to a pump and a discharge portion for infusing a dosage of a pharmaceutical, wherein after implantation the discharge portion of the catheter is in communication with a predetermined infusion site; and coupling the proximal end of the lead to a signal generator; generating an electrical signal with the signal generator to modulate the predetermined site; and operating the pump to discharge the pharmaceutical through the discharge portion of the catheter into the infusion site thereby treating the cognitive impairment and/or disorder.

Other embodiments of the present invention include a system for treating subjects with cognitive impairment and/or disorders. The therapeutic system comprises an electrical stimulation lead that is implanted into the subject's brain. The electrical stimulation lead comprises at least one electrode that is in communication with a predetermined site and delivers electrical signals to the predetermined site in response to received signals; and a signal generator that generates signals for transmission to the electrodes of the lead resulting in delivery of electrical signals to predetermined site thereby treating the cognitive impairment and/or disorder. The electrical stimulation lead may comprise one electrode or a plurality of electrodes in or around the target area. Still further, the signal generator is implanted in the subject's body.

Another example of a therapeutic system is a catheter having a proximal end coupled to a pump and a discharge portion for infusing a dosage of a pharmaceutical, wherein after implantation the discharge portion of the catheter is in communication with a predetermined stimulation site; and a pump to discharge the pharmaceutical through the discharge portion of the catheter into the predetermined stimulation site thereby treating the cognitive impairment and/or disorder.

Still further, another therapeutic system comprises a device that is surgically implanted into the subject such that the device is in communication with a predetermined site. An exemplary device includes a microstimulator (i.e., Bion™ manufactured by Advanced Bionics Corporation) in which the device contains a generating portion and at least one electrode in a single unit. In further embodiments, a lead assembly is associated with at least one electrode of the microstimulator such that the lead can stimulate the predetermined site not in direct contact with the microstimulator.

Other therapeutic systems include a probe that is in communication with the predetermined site and a device that stimulates the probe thereby treating the cognitive impairment and/or disorder. The probe can be, for example, an electrode assembly (i.e., electrical stimulation lead), pharmaceutical-delivery assembly (i.e., catheters) or combinations of these (i.e., a catheter having at least one electrical stimulation lead). The probe is coupled to the device, for example, electrical signal source, pharmaceutical delivery pump, or both which, in turn, is operated to stimulate the predetermined treatment site.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIGS. 1A and 1B illustrate example electrical stimulation systems.

FIGS. 2A-2D illustrate example electrical stimulation leads that may be used in the present invention.

FIG. 3 is a flowchart describing the general procedure.

FIG. 4 shows DBS electrode placement in the subgenual cingulate white matter. Row 1: Sagittal (left, A) and coronal (right, B) views of the subgenual cingulate target (filled circles) localized on the Schaltenbrandt neurosurgical atlas. Row 2: Sagittal (C) and coronal (D) views of the DBS target mapped on a high resolution T1 MRI scans for one patient. Row 3: Sagittal (E) and coronal (F) views of post-op MRI scans demonstrating the location of electrodes for a single subject with the ventral contact centered within the pre-determined location. Abbreviations: sgCg: subgenual cingulate; cc: corpus callosum; g: genu of the corpus callosum; ac: anterior commissure; white circles: electrode target in sgCg white matter; white and black arrows: sgCg gyrus; dotted line: anterior-posterior position of the electrode relative to the ac-g line.

### DETAILED DESCRIPTION OF THE INVENTION

It is readily apparent to one skilled in the art that various embodiments and modifications can be made to the invention disclosed in this Application without departing from the scope and spirit of the invention.

I. Defintions

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms. Some embodiments of the invention may consist of or consist essentially of one or more elements, method steps, and/or methods of the invention. It is contemplated that any method or composition described herein can be implemented with respect to any other method or composition described herein.

As used herein the term “affective disorders” refers to a group of disorders that are commonly associated with co-morbidity of depression and anxiety symptoms.

As used herein the term “anxiety” refers to an uncomfortable and unjustified sense of apprehension that may be diffuse and unfocused and is often accompanied by physiological symptoms.

As used herein the term “anxiety disorder” refers to or connotes significant distress and dysfunction due to feelings of apprehension, guilt, fear, etc. Anxiety disorders include, but are not limited to panic disorders, posttraumatic stress disorder, obsessive-compulsive disorder and phobic disorders.

As used herein, the term “cognitive impairment” refers to an acquired deficit in one or more of memory function, problem solving, orientation and/or abstraction that impinges on an individual's ability to function independently.

As used herein, the term “dementia” refers to a global deterioration of intellectual functioning in clear consciousness, and is characterized by one or more symptoms of disorientation, impaired memory, impaired judgment, and/or impaired intellect.

As used herein, the term “apathy” refers to a slowing of cognitive processes and/or a lack of motivation as manifested by one or more of the following: lack of productivity, lack of initiative, lack of perseverance, diminished socialization or recreation, lack of interest in learning new things, lack of interest in new experiences, lack of emotional responsivity to positive or negative events, unchanging or flat affect, and/or absence of excitement or emotional intensity.

As used herein, the term “enhancing cognitive functions” refers to increasing or improving a patient's normal level of cognitive functioning, including, for example, learning and recall of newly learned information.

As used herein, the term “Brodmann area 25” refers to the defined area of Brodmann area 25 as known by one of skill in the art, as well as the surrounding or adjacent white matter tracts leading to and from Brodmann area 25 and/or white matter tracts that are contiguous with Brodmann area 25. The surrounding or adjacent white matter can include up to approximately a 1 cm radius of Brodmann area 25.

As used herein, the term “Brodmann area 24” refers to the defined area of Brodmann area 24 as known by one of skill in the art, as well as the surrounding or adjacent white matter tracts leading to and from Brodmann area 24 and/or white matter tracts that are contiguous with Brodmann area 24. The surrounding or adjacent white matter can include up to approximately a 1 cm radius of Brodmann area 24.

As used herein, the term “Brodmann area 9” refers to the defined area of Brodmann area 9 as known by one of skill in the art, as well as the surrounding or adjacent white matter tracts leading to and from Brodmann area 9 and/or white matter tracts that are contiguous with Brodmann area 9. The surrounding or adjacent white matter can include up to approximately a 1 cm radius of Brodmann area 9.

As used herein, the term “Brodmann area 10” refers to the defined area of Brodmann area 10 as known by one of skill in the art, as well as the surrounding or adjacent white matter tracts leading to and from Brodmann area 10 and/or white matter tracts that are contiguous with Brodmann area 10. The surrounding or adjacent white matter can include up to approximately a 1 cm radius of Brodmann area 10.

As used herein the term “depression” refers to a morbid sadness, dejection, or melancholy.

As used herein, the term “in communication” refers to one or more electrical stimulation leads and/or catheters being adjacent, in the general vicinity, in close proximity, or directly next to, or in direct contact or directly in the predetermined stimulation site. Thus, one of skill in the art understands that the one or more electrical stimulation leads and/or catheters are “in communication” with the predetermined site of the brain if the stimulation results in a modulation of neuronal activity associated with a site. Still further, “in communication” with brain tissue encompasses surrounding or adjacent white matter tracts or fibers leading to and from the brain tissue and/or white matter tracts or fibers that are contiguous with the brain tissue.

As used herein the term “limbic system” encompasses the amygdala, hippocampus, septum, cingulate gyrus, cingulate cortex, hypothalamus, epithalamus, anterior thalamus, mammillary bodies, and fornix. The limbic system has connections throughout the brain, more particularly with the primary sensory cortices, including the rhinencephalon for smell, the autonomic nervous system via the hypothalamus, and memory areas. Yet further, the limbic system is involved in mood, emotion and thought.

As used herein the term “mania” or “manic” refers to a disordered mental state of extreme excitement.

As used herein the term “mood” refers to an internal emotional state of a person.

As used herein the term “mood disorder” is typically characterized by pervasive, prolonged, and disabling exaggerations of mood and affect that are associated with behavioral, physiologic, cognitive, neurochemical and psychomotor dysfunctions. The major mood disorders include, but are not limited to major depressive disorder (also known as unipolar disorder), bipolar disorder (also known as manic depressive illness or bipolar depression), dysthymic disorder. Other mood disorders may include, but are not limited to major depressive disorder, psychotic; major depressive disorder, melancholic; major depressive disorder, seasonal pattern; postpartum depression; brief recurrent depression; late luteal phase dysphoric disorder (premenstrual dysphoria); and cyclothymic disorder.

As used herein, the term “memory dysfunction” refers to loss or impairment of memory. Memory systems can be divided into four groups episodic memory, semantic memory, procedural memory or working memory, which are further described by Budson and Price (NEJM 2005:352:692-698, which is incorporated herein by reference). Disorders can disrupt these memory systems for example disorder of episodic memory include, but are not limited to Alzheimer's disease, mild cognitive impairment, dementia with Lewy bodies, encephalitis, frontal variant of frontotemporal demential, Korsakoff's syndrome, transient global amnesia, concussion, traumatic brain injury, seizure, hypoxic-ischemic injury, cardiopulmonary bypass, side effects of medication, deficiency of vitamin B12, hypoglycemia, anxiety, temporal-lobe surgery, vascular dementia, and multiple sclerosis. Disorders that disrupt semantic memory can include, Alzheimer's disease, semantic dementia, traumatic brain injury, encephalitis. Disorders that disrupt procedural memory can include Parkinson's disease, Huntiongton's disease, progressive supranuclear palsy, olivopontocerebellar degeneration, depression, and obsessive-compulsive disorder. Disorders that disrupt working memory include normal aging, vascular dementia, frontal variant of frontotemporal dementia, Alzheimer's disease, dementia with Lewy bodies, multiple sclerosis, traumatic brain injury, side effects of medication, attention deficit-hyperactivity disorder, obsessive-compulsive disorder, schizophrenia, Parkinson's disease, Huntington's disease, progressive supranuclear palsy, cardiopulmonary bypass, and deficiency of vitamin B12.

As used herein the term “modulate” refers to the ability to regulate positively or negatively neuronal activity. Thus, the term modulate can be used to refer to an increase, decrease, masking, altering, overriding or restoring neuronal activity. Modulation of neuronal activity affects psychological and/or psychiatric activity of a subject.

As used herein, the term “neuronal” refers to a neuron which is a morphologic and functional unit of the brain, spinal column, and peripheral nerves.

As used herein, the term “pharmaceutical” refers to a chemical or agent that is used as a drug. Thus, the term pharmaceutical and drug are interchangeable.

As used herein, the term “stimulate” or “stimulation” refers to electrical, chemical, and/or magnetic stimulation that modulates the predetermined sites in the brain.

As used herein, the term “subcallosal area” includes the medial gray matter and white matter under the corpus callosum, as well as the white matter tracts that are associated with the subcallosal area. Associated white matter tracts includes the surrounding or adjacent white matter tracts leading to or from a subcallosal area or white matter tracts that are contiguous with the subcallosal area. For the purposes of the present invention, the subcallosal area includes the following gray matter and the white matter tracts, as well as the white matter tracts that are associated with or leading to or from the following areas: subgenual cingulate area, subcallosal gyrus area, ventral/medial prefrontal cortex area, ventral/medial white matter, Brodmann area 24, Brodmann area 25, and/or Brodmann area 10. The surrounding or adjacent white matter tracts can include up to approximately a 1 cm radius of the subcallosal area.

As used herein, the term “subgenual cingulate area” includes the gray matter and white matter tracts associated with the subgenual cingulate area, the white matter tracts that surround or adjacent to the subgenual cingulate area, or the white matter tracts that lead to or from the subgenual cingulate area. The subgenual cingulate area includes Brodmann area 10, Brodmann area 24 and Brodmann area 25. The surrounding or adjacent white matter can include up to approximately a 1 cm radius of the subgenual cingulate area.

As used herein, the term “treating” and “treatment” refers to modulating certain areas of the brain so that the subject has an improvement in the disease, for example, beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. One of skill in the art realizes that a treatment may improve the disease condition, but may not be a complete cure for the disease.

II. Electrical Stimulation Devices

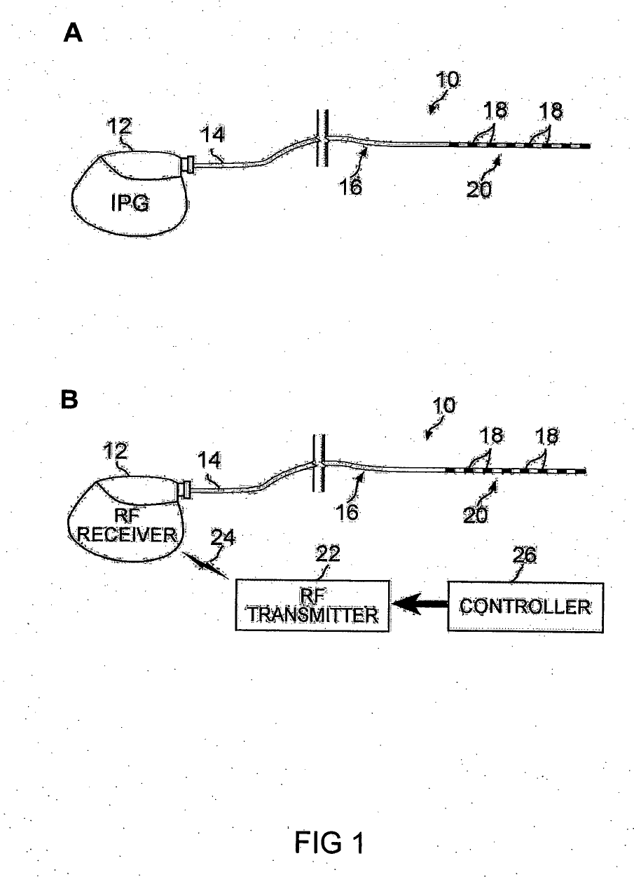

FIGS. 1A and 1B illustrate example electrical stimulation systems or devices 10 used to provide deep brain stimulation. Stimulation system 10 generates and applies a stimulus to a target area of the brain. In general terms, stimulation system 10 includes an implantable pulse generating source, such as an electrical stimulation source 12, and an implantable electrode, for example an electrical stimulation lead 14. In operation, both of these primary components are implanted in the person's body. Stimulation source 12 is coupled to a connecting portion 16 of electrical stimulation lead 14. Stimulation source 12 controls the electrical signals transmitted to electrodes 18 located on a stimulating portion 20 of electrical stimulation lead 14, located adjacent the target brain tissue, according to suitable signal parameters (i.e., duration, intensity, frequency, etc.). A doctor, the patient, or another user of stimulation source may directly or indirectly input signal parameters for controlling the nature of the electrical stimulation provided.

Another exemplary stimulation system or device includes a microstimulator (i.e., Bion™, manufactured by Advanced Bionics Corporation) in which the device contains a signal generating portion and at least one electrode in a the same unit or single unit, as defined in U.S. Pat. Nos. 6,051,017; 6,735,475 and 6,735,474, each of which are incorporated herein in its entirety. In further embodiments, a lead assembly is associated with at least one electrode of the microstimulator such that the lead can stimulate the predetermined site not in contact with the micro stimulator.

In one embodiment, as shown in FIG. 1A, stimulation source 12 includes an implantable pulse generator (IPG). One of skill in the art is aware that any commercially available implantable pulse generator can be used in the present invention, as well as a modified version of any commercially available pulse generator. Thus, one of skill in the art would be able to modify an IPG to achieve the desired results. An exemplary IPG is one that is manufactured by Advanced Neuromodulation Systems, Inc., such as the Genesis® System, part numbers 3604, 3608, 3609, and 3644. Another example of an IPG is shown in FIG. 1B, which shows stimulation source 12 including an implantable wireless receiver. An example of a wireless receiver may be one manufactured by Advanced Neuromodulation Systems, Inc., such as the Renew® System, part numbers 3408 and 3416. The wireless receiver is capable of receiving wireless signals from a wireless transmitter 22 located external to the person's body. The wireless signals are represented in FIG. 1B by wireless link symbol 24. A doctor, the patient, or another user of stimulation source 12 may use a controller 26 located external to the person's body to provide control signals for operation of stimulation source 12. Controller 26 provides the control signals to wireless transmitter 22, wireless transmitter 22 transmits the control signals and power to the wireless receiver of stimulation source 12, and stimulation source 12 uses the control signals to vary the signal parameters of electrical signals transmitted through electrical stimulation lead 14 to the stimulation site. An example wireless transmitter may be one manufactured by Advanced Neuromodulation Systems, Inc., such as the Renew® System, part numbers 3508 and 3516.

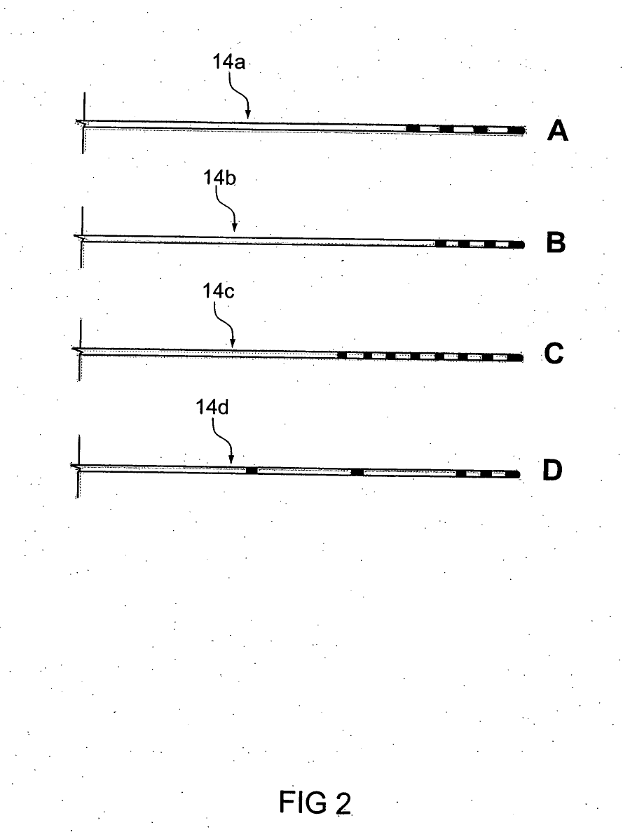

FIGS. 2A through 2D illustrate example electrical stimulation leads 14 that may be used to provide electrical stimulation to an area of the brain, however, one of skill in the art is aware that any electrical lead may be used in the present invention. As described above, each of the one or more leads 14 incorporated in stimulation system 10 includes one or more electrodes 18 adapted to be positioned near the target brain tissue and used to deliver electrical stimulation energy to the target brain tissue in response to electrical signals received from stimulation source 12. A percutaneous lead 14, such as example leads shown in FIG. 2A-2D, includes one or more circumferential electrodes 18 spaced apart from one another along the length of lead 14. Circumferential electrodes 18 emit electrical stimulation energy generally radially in all directions.

III. Implantation of Electrical Stimualtion Devices

While not being bound by the description of a particular procedure, patients who are to have an electrical stimulation lead or electrode implanted into the brain, generally, first have a stereotactic head frame, such as the Leksell, CRW, or Compass, mounted to the patient's skull by fixed screws. However, frameless techniques may also be used. Subsequent to the mounting of the frame, the patient typically undergoes a series of magnetic resonance imaging sessions, during which a series of two dimensional slice images of the patient's brain are built up into a quasi-three dimensional map in virtual space. This map is then correlated to the three dimensional stereotactic frame of reference in the real surgical field. In order to align these two coordinate frames, both the instruments and the patient must be situated in correspondence to the virtual map. The current way to do this is to rigidly mount the head frame to the surgical table. Subsequently, a series of reference points are established to relative aspects of the frame and the patient's skull, so that either a person or a computer software system can adjust and calculate the correlation between the real world of the patient's head and the virtual space model of the patient's MRI scans. The surgeon is able to target any region within the stereotactic space of the brain with precision (i.e., within 1 mm). Initial anatomical target localization is achieved either directly using the MRI images, or indirectly using interactive anatomical atlas programs that map the atlas image onto the stereotactic image of the brain. As is described in greater detail below, the anatomical targets may be stimulated directly or affected through stimulation in another region of the brain.

Stimulation of the subgenual cingulate area results in blood flow changes in other areas of the brain, for example other areas associated with the limbic-cortical system. See for example areas described in Mayberg et al. (Neuron, 45:1-10, 2005); U.S. patent application 20050033379A1, and U.S. Provisional application No. 60/567332, each of which is incorporated herein by reference in its entirety. Thus, it is within the purview of one of skill in the art to stimulate these identified areas, as well as the subgenual cingulate area, or any gray and/or white matter associated with the identified areas, more specifically, white matter tracts afferent to or efferent from the abovementioned brain tissue.

In certain embodiments, the predetermined site or target area can include but are limited to the subgenual cingulate area, hypothalamus, orbital frontal cortex, anterior insula, medial frontal cortex, dorsolateral prefrontal cortex, dorsal anterior cortex, posterior cingulate area, premotor cortex, orbital frontal cortex, parietal region, ventrolateral prefrontal cortex, dorsal cingulate, dorsal anterior cingulate, caudate nucleus, anterior thalamus, nucleus accumbens; periaqueductal gray area; brainstem; and/or the surrounding or adjacent white matter tracts leading to or from the all of these listed areas or white matter tracts that are contiguous. Thus, stimulation of any of the above brain tissue areas, as well as any white matter tracts afferent to or efferent from the abovementioned brain tissue can result in alterations or changes that alleviate or improve the affective disorder of the subject.

Based upon the coordinates derived or described above, the electrical stimulation lead 14 can be positioned in the brain. Typically, an insertion cannula for electrical stimulation lead 14 is inserted through the burr hole into the brain, but a cannula is not required. For example, a hollow needle may provide the cannula. The cannula and electrical stimulation lead 14 may be inserted together or lead 14 may be inserted through the cannula after the cannula has been inserted.

Once an electrical stimulation lead, such as lead 14, has been positioned in the brain, the lead is uncoupled from any stereotactic equipment present, and the cannula and stereotactic equipment are removed. Where stereotactic equipment is used, the cannula may be removed before, during, or after removal of the stereotactic equipment. Connecting portion 16 of electrical stimulation lead 14 is laid substantially flat along the skull. Where appropriate, any burr hole cover seated in the burr hole may be used to secure electrical stimulation lead 14 in position and possibly to help prevent leakage from the burr hole and entry of contaminants into the burr hole. Example burr hole covers that may be appropriate in certain embodiments are illustrated and described in co-pending U.S. Application Nos. 60/528,604 and 60/528,689, both filed Dec. 11, 2003 and entitled “Electrical Stimulation System and Associated Apparatus for Securing an Electrical Stimulation Lead in Position in a Person's Brain”, each of which are incorporated herein in its entirety.

Once electrical stimulation lead 14 has been inserted and secured, connecting portion 16 of lead 14 extends from the lead insertion site to the implant site at which stimulation source 12 is implanted. The implant site is typically a subcutaneous pocket formed to receive and house stimulation source 12. The implant site is usually positioned a distance away from the insertion site, such as near the chest, below the clavicle or alternatively near the buttocks or another place in the torso area. Once all appropriate components of stimulation system are implanted, these components may be subject to mechanical forces and movement in response to movement of the person's body. A doctor, the patient, or another user of stimulation source may directly or indirectly input signal parameters for controlling the nature of the electrical stimulation provided.

Although example steps are illustrated and described, the present invention contemplates two or more steps taking place substantially simultaneously or in a different order. In addition, the present invention contemplates using methods with additional steps, fewer steps, or different steps, so long as the steps remain appropriate for implanting an example stimulation system into a person for electrical stimulation of the person's brain.

IV. Infusion Pump

In further embodiments, it may be desirable to use a drug delivery system independent of or in combination with electrical stimulation of the brain. Drug delivery may be used independent of or in combination with a lead/electrode to provide electrical stimulation and chemical stimulation. When used, the drug delivery catheter is implanted such that the proximal end of the catheter is coupled to a pump and a discharge portion for infusing a dosage of a pharmaceutical or drug. Implantation of the catheter can be achieved by combining data from a number of sources including CT, MRI or conventional and/or magnetic resonance angiography into the stereotactic targeting model. Thus, without being bound to a specific procedure, implantation of the catheter can be achieved using similar techniques as discussed above for implantation of electrical leads, which is incorporated herein. The distal portion of the catheter can have multiple orifices to maximize delivery of the pharmaceutical while minimizing mechanical occlusion. The proximal portion of the catheter can be connected directly to a pump or via a metal, plastic, or other hollow connector, to an extending catheter.

Any type of infusion pump can be used in the present invention. For example, “active pumping” devices or so-called peristaltic pumps are described in U.S. Pat. Nos. 4,692,147, 5,840,069, and 6,036,459, which are incorporated herein by reference in their entirety. Peristaltic pumps are used to provide a metered amount of a drug in response to an electronic pulse generated by control circuitry associated within the device. An example of a commercially available peristaltic pump is SynchroMed® implantable pump from Medtronic, Inc., Minneapolis, Minn.

Other pumps that may be used in the present invention include accumulator-type pumps, for example certain external infusion pumps from Minimed, Inc., Northridge, Calif. and Infusaid® implantable pump from Strato/Infusaid, Inc., Norwood, Mass. Passive pumping mechanisms can be used to release an agent in a constant flow or intermittently or in a bolus release. Passive type pumps include, for example, but are not limited to gas-driven pumps described in U.S. Pat. Nos. 3,731,681 and 3,951,147; and drive-spring diaphragm pumps described in U.S. Pat. Nos. 4,772,263, 6,666,845, 6,620,151 which are incorporated by reference in its entirety. Pumps of this type are commercially available, for example, Model 3000® from Arrow International, Reading, Pa. and IsoMed® from Medtronic, Inc., Minneapolis, Minn.; AccuRx® pump from Advanced Neuromodulation Systems, Inc., Plano, Tex.

Instances in which chemical and electrical stimulation will be administered to the subject, a catheter having electrical leads may be used, similar to the ones described in U.S. Pat. Nos. 6,176,242; 5,423,877; 5,458,631 and 5,119,832, each of which are incorporated herein by reference in its entirety.

V. Identifying a Subject with Cognitive Impairment

Subjects to be treated using the present invention can be selected, identified and/or diagnosed based upon the accumulation of physical, chemical, and historical behavioral data on each patient. One of skill in the art is able to perform the appropriate examinations to accumulate such data. One type of examination can include neurological examinations, which can include mental status evaluations, which can further include a psychiatric assessment. Other types of examinations can include, but are not limited to, motor examination, cranial nerve examination, cognitive assessment and neuropsychological tests (i.e., Minnesota Multiphasic Personality Inventory, Beck Depression Inventory, or Hamilton Rating Scale for Depression).

In addition to the above examinations, imaging techniques can be used to determine normal and abnormal brain function that can result in disorders. Functional brain imaging allows for localization of specific normal and abnormal functioning of the nervous system. This includes electrical methods such as electroencephalography (EEG), magnetoencephalography (MEG), single photon emission computed tomography (SPECT), as well as metabolic and blood flow studies such as functional magnetic resonance imaging (fMRI), and positron emission tomography (PET) which can be utilized to localize brain function and dysfunction.

VI. Treatment of an Cognitive Impairment or Enhancement of Cognitive Abilities

Initially, there is an impetus to treat psychiatric disorders with direct modulation of activity in that portion of the brain causing the pathological behavior. In this regard, there have been a large number of anatomical studies that have helped to identify the neural structures and their precise connections which are implicated in psychiatric activity/disorders. These are the structures that are functioning abnormally and manifesting in psychiatric/behavioral/addiction disorders. Numerous anatomical studies from autopsies, animal studies, and imaging such as computerized tomography (CT) scans, and magnetic resonance imaging (MRI) scans have demonstrated the role of these structures and their connections in psychiatric activity/disorders. In addition to these anatomical studies, a number of physiological techniques and diagnostic tools are used to determine the physiological aberrations underlying these disorders. This includes electrical methods such as electroencephalography (EEG), magnetoencephalography (MEG), as well as metabolic and blood flow studies such as functional magnetic resonance imaging (fMRI), and positron emission tomography (PET). The combination of the anatomical and physiological studies have provided increased insight into our understanding of the structures which are involved in the normal functioning or activity of the brain and the abnormal functioning manifesting in psychiatric, behavioral and addiction disorders.

Accordingly, the present invention relates to modulation of neuronal activity to affect psychological or psychiatric activity and/or mental activity. The present invention finds particular application in the modulation of neuronal function or processing to effect a functional outcome. The modulation of neuronal function is particularly useful with regard to the prevention, treatment, or amelioration of psychiatric, psychological, conscious state, behavioral, mood, mental activity, cognitive ability, memory and thought activity (unless otherwise indicated these will be collectively referred to herein as “psychological activity” or “psychiatric activity” or “mental activity”). When referring to a pathological or undesirable condition associated with the activity, reference may be made to “psychiatric disorder” or “psychological disorder” instead of psychiatric or psychological activity. Although the activity to be modulated usually manifests itself in the form of a disorder such as a mood disorder (i.e., major depressive disorder, bipolar disorder, and dysthymic disorder) or an anxiety disorder (i.e., panic disorder, posttraumatic stress disorder, obsessive-compulsive disorder and phobic disorder), or cognitive disorders (dementia, etc.) it is to be appreciated that the invention may also find application in conjunction with enhancing or diminishing any neurological or psychiatric function, not just an abnormality or disorder. Psychiatric activity that may be modulated can include, but not be limited to, normal functions such as alertness, conscious state, drive, fear, anger, anxiety, euphoria, sadness, and the fight or flight response.

Thus, in certain embodiments, the present invention can be used to enhance or improve cognitive abilities in a subject suffering from cognitive impairments. Such impairments are associated with mild cognitive impairment (MCI), Alzheimer's disease, dementia, post irradiation cognitive impairment, drug-induced depression of cognitive function, cognitive impairment associated with drug use, drug abuse, medication use, epilepsy, hypoxia, anoxia, hypoglycemia, hyperglycemia, post-stoke, post-head injury, metabolic disorders, other psychiatric disorders, movement disorders (e.g., Parkinson's disease, dystonia, chorea, tics and myoclonus). Other forms of cognitive impairment can include those described by Budson and Price in NEJM 2005:352: 692-698, which is incorporated herein by reference can also be treated.

Still further, the method and system of the present invention can be used to improve motivation, attention, concentration and reward. Thus, stimulation of the predetermined site, for example, the subcallosal area, may be useful to treat attention deficit disorders, drug addiction, disorders of verbal fluency, aphasias, dysphasias, psychomotor retardation, and risk-taking behavior.

Yet further, the stimulation method of the present invention may also be used to effect sleep and appetite, libido, neuroendocrine function and memory. Thus, the present invention can be used to treat disorders associated with these functions.

The present invention finds particular utility in its application to human psychological or psychiatric activity/disorder or cognitive activity/disorder. However, it is also to be appreciated that the present invention is applicable to other animals which exhibit behavior that is modulated by the brain. This may include, for example, rodents, primates, canines, felines, elephants, dolphins, etc. Utilizing the various embodiments of the present invention, one skilled in the art may be able to modulate the functional outcome of the brain to achieve a desirable result.

One technique that offers the ability to affect neuronal function is the delivery of electrical, chemical, and/or magnetic stimulation for neuromodulation directly to target tissues via an implanted device having a probe. The probe can be a stimulation lead or electrode assembly or drug-delivery catheter, or any combination thereof. The electrode assembly may be one electrode, multiple electrodes, or an array of electrodes in or around the target area. The proximal end of the probe can be coupled to a device, such as an electrical signal source, pharmaceutical delivery pump, or both which, in turn, is operated to stimulate the predetermined treatment site. In certain embodiments, the probe can be incorporated into the device such that the probe and the signal generating device are a single unit.

Certain embodiments of the present invention involve a method of treating a cognitive impairment and/or disorder comprising the steps of: surgically implanting an electrical stimulation lead having a proximal end and a stimulation portion, wherein after implantation the stimulation portion is in communication with a predetermined site; coupling the proximal end of the lead to a signal generator; and generating an electrical signal with the signal generator to modulate the predetermined site thereby treating the cognitive impairment and/or disorder.

In further embodiments, neuromodulation of the predetermined site of the present invention can be achieved using magnetic stimulation. One such system that can be employed and that is well known in the art is described in U.S. Pat. No. 6,425,852, which is incorporated herein by reference in its entirety.

The therapeutic system or deep brain stimulation system of the present invention is surgically implanted as described in the above sections. One of skill in the art is cognizant that a variety of electrodes or electrical stimulation leads may be utilized in the present invention. It is desirable to use an electrode or lead that contacts or conforms to the target site for optimal delivery of electrical stimulation. One such example, is a single multi contact electrode with eight contacts separated by 2½ mm each contract would have a span of approximately 2 mm. Another example is an electrode with two 1 cm contacts with a 2 mm intervening gap. Yet further, another example of an electrode that can be used in the present invention is a 2 or 3 branched electrode/catheter to cover the predetermined site or target site. Each one of these three pronged catheters/electrodes have four contacts 1-2 mm contacts with a center to center separation of 2 of 2.5 mm and a span of 1.5 mm. Similar designs with catheters to infuse drugs with single outlet pore at the extremities of these types of catheters or along their shaft may also be designed and used in the present invention.

Still further, the present invention extends to methods of transplanting cells into a predetermined site to treat cognitive impairment and/or disorders. It is envisioned that the transplanted cells can replace damaged, degenerating or dead neuronal cells, deliver a biologically active molecule to the predetermined site or to ameliorate a condition and/or to enhance or stimulate existing neuronal cells. Such transplantation methods are described in U.S. Application No. US20040092010, which is incorporated herein by reference in its entirety.

Cells that can be transplanted can be obtained from stem cell lines (i.e., embryonic stem cells, non-embryonic stem cells, etc.) and/or brain biopsies, including tumor biopsies, autopsies and from animal donors. (See U.S. Application No. US20040092010; U.S. Pat. Nos. 5,735,505 and 6,251,669; Temple, Nature Reviews 2:513-520 (2000); Bjorklund and Lindvall, Nat. Neurosci. 3:537-544 (2000)), each of which is incorporated herein by reference in its entirety). Brain stem cells can then be isolated (concentrated) from non-stem cells based on specific “marker” proteins present on their surface. In one such embodiment, a fluorescent antibody specific for such a marker can be used to isolate the stem cells using fluorescent cell sorting (FACS). In another embodiment an antibody affinity column can be employed. Alternatively, distinctive morphological characteristics can be employed.

Alternatively, affective disorders can be treated by utilizing other known methods to alter the neuronal activity of the predetermined sites. For example, lesioning and mechanical disruption can be used as described by U.S. Pat. Nos. 6,629,973, 3,653,385, which is incorporated herein by reference in its entirety.

The predetermined site or target area can include but are limited to the subcallosal area, subgenual cingulate area, hypothalamus, orbital frontal cortex, anterior insula, medial frontal cortex, dorsolateral prefrontal cortex, dorsal anterior cortex, posterior cingulate area, premotor cortex, orbital frontal cortex, parietal region, ventrolateral prefrontal cortex, dorsal cingulate, dorsal anterior cingulate caudate nucleus, anterior thalamus, nucleus accumbens; frontal pole periaqueductal gray area; brainstem; and/or the surrounding or adjacent white matter tracts leading to or from the all of these listed areas or white matter tracts that are contiguous. Thus, stimulation of any of the above brain tissue areas, as well as any white matter tracts afferent to or efferent from the abovementioned brain tissue can result in alterations or changes that alleviate or improve the affective disorder of the subject.

Still further, Bordmann areas that may be stimulated include Brodmann area 25, Brodmann area 10, Brodmann area 31, Brodmann area 9, Brodmann area 24b, Brodmann area 47, Brodmann area 32/Brodmann area 10, Brodmann area 24, Brodmann area 46, Brodmann area 6, Brodmann area 32/Brodmann area 11, Brodmann area 11/Brodmann area 10, Brodmann area 46/Brodmann area 9, and Brodmann area 39. Thus, stimulation of any of the above brain tissue areas, as well as any white matter tracts afferent to or efferent from the abovementioned brain tissue can result in alterations or changes that alleviate or improve the affective disorder of the subject.

In certain embodiments, the predetermined site or target area is a subcallosal area, more preferably, the subgenual cingulate area, and more preferably Brodmann area 25/Brodmann area 24. Stimulation of a subcallosal area (i.e., subgenual cingulate area or Brodmann area 25/Brodmann area 24) and/or the surrounding or adjacent white matter tracts leading to or from the subcallosal area or white matter tracts that are contiguous with the subcallosal area results in changes that alleviate or improve the cognitive impairment of the subject. It is contemplated that modulating a subcallosal area, more particularly a subgenual cingulate area, can result in increasing, decreasing, masking, altering, overriding or restoring neuronal activity resulting in treatment of the cognitive impairment and/or disorder or enhancing cognition. Yet further stimulation of a subgenual cingulate area, more particularly Brodman area 25, results in modulation of neuronal activity of other areas of the brain, for example, Brodmann area 9, Brodmann area 10, Brodmann area 24, the hypothalamus, and the brain stem.

Using the therapeutic stimulation system of the present invention, the predetermined site or target area is stimulated in an effective amount or effective treatment regimen to decrease, reduce, modulate or abrogate the cognitive impairment and/or disorder. Thus, a subject is administered a therapeutically effective stimulation so that the subject has an improvement in the parameters relating to the affective disorder including subjective measures such as, for example, neurological examinations and neuropsychological tests (i.e., Minnesota Multiphasic Personality Inventory, Beck Depression Inventory, Mini-Mental Status Examination (MMSE), Hamilton Rating Scale for Depression, Wisconsin Card Sorting Test (WCST), Tower of London, Stroop task, MADRAS, CGI, N-BAC, or Yale-Brown Obsessive Compulsive score (Y-BOCS)), motor examination, and cranial nerve examination, and objective measures including use of additional psychiatric medications, such as anti-depressants, or other alterations in cerebral blood flow or metabolism and/or neurochemistry. The improvement is any observable or measurable improvement. Thus, one of skill in the art realizes that a treatment may improve the patient condition, but may not be a complete cure of the disease.

Treatment regimens may vary as well, and often depend on the health and age of the patient. Obviously, certain types of disease will require more aggressive treatment, while at the same time, certain patients cannot tolerate more taxing regimens. The clinician will be best suited to make such decisions based on the known subject's history.

According to one embodiment of the present invention, the target site is stimulated using stimulation parameters such as, pulse width of about 1 to about 500 microseconds, more preferable, about 1 to about 90 microseconds; frequency of about 1 to about 300 Hz, more preferably, about 100 to about 185 Hz; and voltage of about 0.5 to about 10 volts, more preferably about 1 to about 10 volts. It is known in the art that the range for the stimulation parameters may be greater or smaller depending on the particular patient needs and can be determined by the physician. Other parameters that can be considered may include the type of stimulation for example, but not limited to acute stimulation, subacute stimulation, and/or chronic stimulation.

It is envisioned that stimulation of any of the above mentioned predetermined sites modulates other targets in the limbic-cortical circuit or pathway thereby improving any dysfunctional limbic-cortical circuits resulting in an improvement or alleviation or providing remission of cognitive impairment in the treated subjects. Other such improvements can be sensations of calm, tranquility, peacefulness, increased energy and alertness, improved mood, improvement in attention and thinking, memory, cognitive ability, improvement in motor speed, improvement in mental speed and in spontaneity of speech, improved sleep, improved appetite, improved limbic behavior, increased motivation, decreases in anxiety, decreases in repetitive behavior, impulses, obsessions, etc.

For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether objective or subjective.

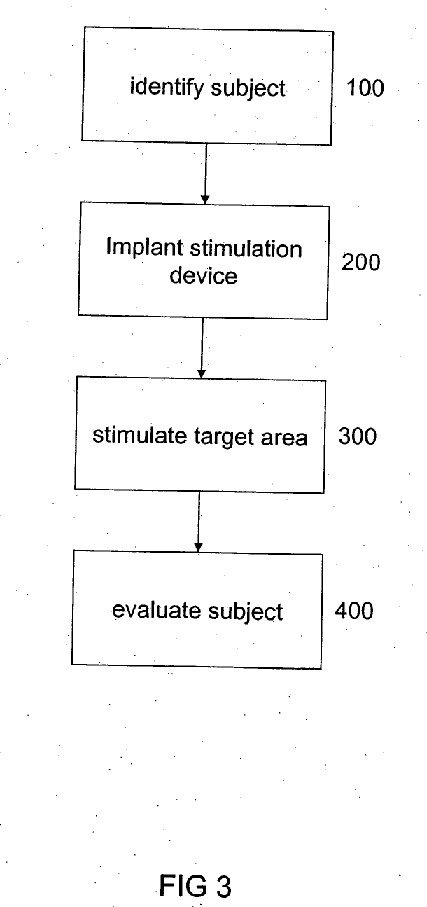

FIG. 3 summarizes the general procedure of the present invention. Any of the above described methods can be used to identify a subject or diagnose a subject that suffers from a cognitive disorder (100). Once the subject is identified, a stimulation device is implanted (200) into the subject such that the predetermined area of the subject's brain is stimulated (300). After the target area has been stimulated (i.e., electrical, chemical, thermal magnetic and/or ultrasonic stimulation), the subject is evaluated to determine the change in the cognitive disorder or enhancement in cognitive ability. One of skill in the art realizes that the present invention is not bound by the described methods or devices and that any method or device that would result in neuromodulation of the predetermined area could be used in the present invention.

VII. Combination Treatment

In order to increase the effectiveness of the electrical stimulation method of the present invention, it may be desirable to combine electrical stimulation with chemical stimulation to treat the cognitive impairment and/or enhance cognitive ability.

In one preferred alternative, an implantable pulse generating source electrode and an implantable pump and catheter(s) are used to deliver electrical stimulation and/or one or more stimulating drugs to the above mentioned areas as a treatment for cognitive impairment and/or disorders and/or enhance cognitive ability.

Herein, stimulating drugs comprise medications, anesthetic agents, synthetic or natural peptides or hormones, neurotransmitters, cytokines and other intracellular and intercellular chemical signals and messengers, and the like. In addition, certain neurotransmitters, hormones, and other drugs are excitatory for some tissues, yet are inhibitory to other tissues. Therefore, where, herein, a drug is referred to as an “excitatory” drug, this means that the drug is acting in an excitatory manner, although it may act in an inhibitory manner in other circumstances and/or locations. Similarly, where an “inhibitory” drug is mentioned, this drug is acting in an inhibitory manner, although in other circumstances and/or locations, it may be an “excitatory” drug. In addition, stimulation of an area herein includes stimulation of cell bodies and axons in the area.

Similarly, excitatory neurotransmitter agonists (i.e., norepinephrine, epinephrine, glutamate, acetylcholine, serotonin, dopamine), agonists thereof, and agents that act to increase levels of an excitatory neurotransmitter(s) (i.e., edrophonium; Mestinon; trazodone; SSRIs (i.e., flouxetine, paroxetine, sertraline, citalopram and fluvoxamine); tricyclic antidepressants (i.e., imipramine, amitriptyline, doxepin, desipramine, trimipramine and nortriptyline), monoamine oxidase inhibitors (i.e., phenelzine, tranylcypromine, isocarboxasid)), generally have an excitatory effect on neural tissue, while inhibitory neurotransmitters (i.e., dopamine, glycine, and gamma-aminobutyric acid (GABA)), agonists thereof, and agents that act to increase levels of an inhibitory neurotransmitter(s) generally have an inhibitory effect. (Dopamine acts as an excitatory neurotransmitter in some locations and circumstances, and as an inhibitory neurotransmitter in other locations and circumstances.) However, antagonists of inhibitory neurotransmitters (i.e., bicuculline) and agents that act to decrease levels of an inhibitory neurotransmitter(s) have been demonstrated to excite neural tissue, leading to increased neural activity. Similarly, excitatory neurotransmitter antagonists (i.e., prazosin, and metoprolol) and agents that decrease levels of excitatory neurotransmitters may inhibit neural activity. Yet further, lithium salts and anesthetics (i.e., lidocane) may also be used in combination with electrical stimulation.

In further embodiments, macrocyclic lactones, and particularly bryostatin-1 can be administered alone or in combination with electrical stimulation. Such compounds are described in U.S. Pat. No. 6,825,229, U.S. Pat. Nos. 6,187,568, 6,043,270, 5,393,897, 5,072,004, 5,196,447, 4,833,257, 4,611,066, and 4,560,774, each of which is incorporated herein by reference in its entirety.

In addition to electrical stimulation and/or chemical stimulation, other forms of stimulation can be used, for example magnetic, or thermal, ultrasonic or combinations thereof. Magnetic stimulation can be provided by internally implanted probes or by externally applied directed magnetic fields, for example, U.S. Pat. Nos. 6,592,509; 6,132,361; 5,752,911; and 6,425,852, each of which is incorporated herein in its entirety. Thermal stimulation can be provided by using implanted probes that are regulated for heat and/or cold temperatures which can stimulate or inhibit neuronal activity, for example, U.S. Pat. No. 6,567,696, which is incorporated herein by reference in its entirety.

### VIII. EXAMPLES

The following examples are included to demonstrate preferred embodiments, more particularly methods and procedures, of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Patients

This pilot study included six patients with treatment resistant major depression (TRD) referred by mood disorder specialists (Table 1). The clinical diagnosis of major depressive disorder, major depressive episode (MDD-MDE) was independently confirmed by two psychiatrists and a research coordinator using the Structured Clinical Interview for DSM-IV (First et al., 2001). Patients were selected for surgery because they were resistant to all available therapeutic options. All had failed to respond to a minimum of four different classes of antidepressant medications, prescribed at maximal tolerable doses. Failed treatments included SSRI, venlafaxine, bupropion, monoamine oxidase inhibitor, and tricyclic antidepressants, as well as augmentation strategies using lithium, atypical antipsychotics, and anticonvulsants. Five of the six patients had received electroconvulsive therapy and all had attempted cognitive behavioral therapy without clinical improvement.

TABLE 1
Patient Demographics

| | Patient# | | | | | | |
|---|---|---|---|---|---|---|---|
| | 1 * | 2 # | 3 * | 4 # | 5 * | 6 * | Group |
| Gender | F | M | F | M | M | F | 3F/3M |
| current age | 48 | 59 | 45 | 48 | 37 | 39 | 46 ± 8 |
| age MDD onset | 18 | 45 | 21 | 40 | 19 | 34 | 29.5 ± 12 |
| Current episode (yrs) | 1.5 | 3 | 6 | 8 | 10 | 5 | 5.6 ± 3 |
| # Lifetime Episodes | 12 | 9 | 3 | 1 | 2 | 1 | 4.7 ± 5 |
| Hamilton Depression | 29 | 20 | 27 | 24 | 26 | 25 | 25 ± 3 |
| score (17 item) | | | | | | | |
| Past ECT | no | yes | Yes | yes | yes | yes | 5 of 6 |
| Past Psychotherapy | yes | yes | Yes | yes | yes | yes | 6 of 6 |
| Family History MDD | yes | No | Yes | yes | yes | yes | 5 of 6 |
| DSM IV Diagnosis | UP | BPII | UP | UP | UP | UP | 5 UP |
| Melancholic subtype | yes | No | Yes | no | yes | yes | 4 of 6 |
| Current Medications\hat{ } | 1-5 | 1, 3 | 1-4 | 1, 2, 4, 6, 7 | 1, 7 | 1, 4, 5, 7 | |

* Response: >50% change in Ham 17 Depression Score at 6 months.
#: Non-resp: <50% change in Ham 17 Depression Score at 6 months.
\hat{ }current medications:
1 = SSRI/SNRI; 2 = bupropion; 3 = atypical antipsychotic 4 = benzodiazepine; 5 = stimulant; 6 = mood stabilizer 7 = other
Abbreviations:
MDD = major depressive disorder;
ECT = electroconvulsive therapy;
DSM IV = Diagnostic and Statistical Manual of Mental Disorders, Version IV;
UP = unipolar;
BP = bipolar

### Example 2

### Surgery

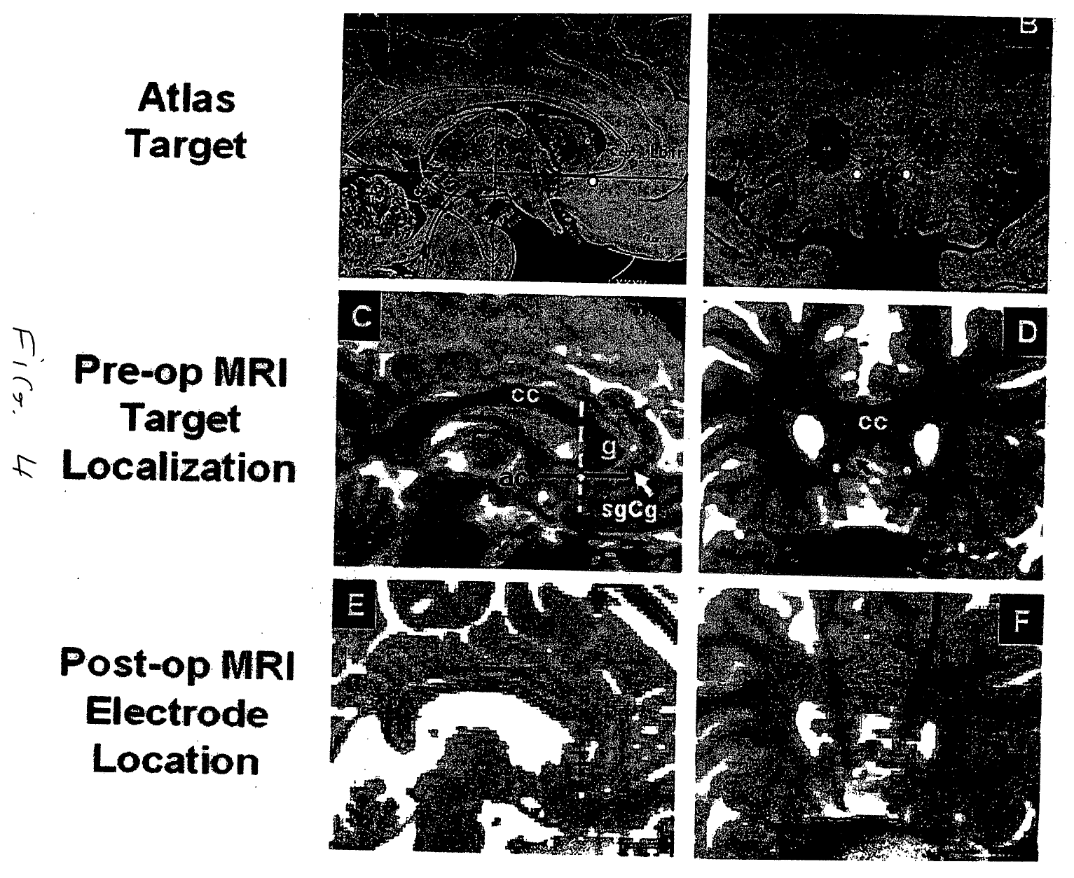

The general surgical procedure for the implantation of DBS electrodes has been previously described (Lang and Lozano, 1998). A stereotactic frame (Leksell G; Elekta, Inc., Atlanta, Ga.) was affixed to the patient's head on the morning of surgery and preoperative MR images were obtained (Signa, 1.5 tesla; General Electric, Milwaukee, Wis.). The x, y, and z coordinates of the anterior (AC) and posterior commissures (PC) were determined using axial 3D T1 MR images. To target the subgenual cingulate white matter target, a midline T2 sagittal image was chosen and the cingulate gyrus below the genu of the corpus callosum was identified (FIG. 4, row 1) (Schaltenbrand and Wahren, 1977). A line was traced from the most anterior aspect (genu) of the corpus callosum to the anterior commissure and the midpoint was selected (FIG. 4, row 2, left). The T2 coronal section correspondent to the plane of this midpoint was identified and the coordinates of the transition between the gray and white matters of area 25 were calculated (FIG. 4, Row 2, right).

In the operating room under local anesthesia, a burr hole was drilled 2 cm from the midline in front of the coronal suture. The underlying dura mater was opened, and the exposed pial surface coagulated. Tisseal (Immuno, Vienna, Austria) was used to prevent cerebrospinal fluid egress and minimize brain shift. The Leksell arc was attached to the head frame and set to the target coordinates. Micro-recordings were started 10 mm above the target using electrodes made from parylene-C-insulated tungsten wires and plated with gold and platinum. Tip lengths ranged from 15 to 40 μm and impedances ranged from 0.2 to 1.5 MΩ. Cell activity was amplified (DAM 80 WPI Instruments) with a gain of 1000 and initially filtered to 0.1-10 kHz. The signal was displayed on an oscilloscope and directed to a window discriminator (Winston Electronics) and an audio monitor (Grass AM 8, with noise clipping circuit). In the present study, microelectrode mapping was mainly used to confirm the anatomic location of the gray and white matters of area 25, characterized respectively by the recording of neuronal activity and cell sparse areas. The transition between these two regions was chosen as the final target for the implantation of the electrodes. Final electrode location was confirmed by post-operative MRI (example, FIG. 4, Row 3).

DBS quadripolar electrodes (Medtronic 3387; Medtronic, Inc., Minneapolis, Minn.) were implanted bilaterally. Each of the 4 electrode contacts was tested for adverse effects and clinical benefits. These contacts were numbered from 0-3 (right hemisphere) and 4-7 (left hemisphere), 0 and 4 being the most ventral and 3 and 7 the most dorsal contacts. The electrodes remained externalized for 5-7 days for clinical testing. They were then connected to a pulse generator (Kinetra, Medtronic, Minneapolis, Minn.) that was implanted in the infraclavicular region under general anesthesia. Prophylactic antibiotics were used for 24 hours after each of the surgical procedures.

The spontaneous report or occurrence of any acute behavioral, cognitive, motor or autonomic effects were sought during blinded, sequential, stimulation of successive, individual contacts (monopolar stimulation, 60 μ-sec pulse-widths, 130 Hz). Voltage was progressively increased up to 9.0 V at each of the 8 electrode contacts (four per side), as tolerated. Voltage was increased by approximately 1.0 V every 30 seconds, with a 15-20 second pause between adjustments, allowing time for patients to identify an effect, if present. Patients reported no motor or sensory phenomena that cued them as to whether current was either on or off.

In response to electrical stimulation at specific contacts and with specific stimulation parameters, all patients spontaneously reported acute effects including ‘sudden calmness or lightness’, ‘disappearance of the void,’ sense of heightened awareness, increased interest and ‘connectedness,’ and sudden brightening of the room including a description of the sharpening of visual details and intensification of colors. Reproducible and reversible changes in these phenomena, time-locked with stimulation, were observed at specific contacts and parameters for individual patients and not with sham or sub-threshold stimulation at those same sites. Increases in motor speed, volume and rate of spontaneous speech and improved prosody were observed. In addition, changes in both positive and negative affective rating scores (PANAS scale) (Watson and Clark, 1988) occurred coincident with the patients' spontaneous statements. There were no overt adverse affective or autonomic changes with stimulation at settings producing these improvements. However, all patients experienced stimulation-dose dependent adverse effects including lightheadedness and psychomotor slowing at high settings (over 7.0 Volts), most often seen at the superior electrode contact.

### Example 3

### Post Operative Findings: Short-Term Stimulation Effects

Post operative MR imaging confirmed the placement of the DBS electrodes within the subgenual cingulate white matter (Cg25WM) bilaterally as targeted. (FIG. 4, row 3: E/F). During the 5 day post-operative period, and prior to placement of the pulse generator, daily short sessions of DBS were used to refine final contact selection and stimulation parameters. Systematic testing of individual and paired unilateral and bilateral contacts was performed with a variety of parameters (monopolar [contact anode; case cathode] and bipolar, pulse width of 30 to 250 microseconds, frequency of 10 Hz to 130 Hz, progressive increase in voltage from 0.0 to 9.0 Volts) as has been previously described for other DBS applications (Benabid, 2003; Davis et al., 1997; Lang and Lozano, 1998). Acute behavioral changes were again observed during these test sessions. Reproducible improvements in interest, motor speed, activity level, and PANAS scores (reduced negative, increased positive scores) were seen during these stimulation sessions generally using the same contacts and parameters that induced effects in the operating room.

### Example 4

### Post-Operative Selection of Stimulation Parameters

Patients were discharged home with stimulation “off” following implantation of the pulse generators. One week later, chronic DBS was initiated using the lowest voltage and specific electrode contacts that had previously produced acute behavioral effects. Parameters of stimulation were reassessed at weekly intervals with minor adjustments in voltage made to optimize clinical effects. Following a 4 week period of parameter optimization, settings generally remained stable for the remainder of the 6-month follow-up period. The mean stimulation parameters used in this group at 6 months were 4.0 Volts, 60 μ-sec pulse-widths, at a frequency of 130 Hz.

### Example 5

### Clinical Evaluation and Follow-Up

Clinical efficacy was evaluated using standardized ratings by the study psychiatrist blinded to the current stimulus parameters and/or changes. Standardized Ratings included the Hamilton Depression Rating Scale (HDRS-17 and 24 item versions) (Frank, et al., 1991), the Montgomery Asberg Depression Scale (MADRS) (Montgomery and Asberg, 1979), the Clinical Global Impressions Scale (CGI) (National Institute of Mental Health, 1970) and the Positive and Negative Affective Scale (PANAS) (Watson and Clark 1988). (Table 3). Ratings were performed weekly for the first 3 months and biweekly until the study endpoint at 6 months, following baseline assessments at enrollment and 1 week prior to surgery. Medications were unchanged throughout the 6 month follow-up period.

Standard criteria for antidepressant response and remission were applied (Frank et al., 1991). Response was defined as a decrease in the HDRS-17 score of 50% or greater from the pretreatment baseline; remission as an absolute HDRS-17 score <8. One month post-op, two patients met criteria for clinical response (Table 2). By 2 months, 5 of the six patients met the defined response threshold. Continued antidepressant response was seen in 4 of these subjects, with some variability up to 5 months. At the 6 month study endpoint, antidepressant response was maintained in four subjects (66%). Moreover, 3 of these subjects achieved remission or near remission of illness. Consistent with the improvements seen in the HDRS-17 scores, comparable changes were also demonstrated on other quantitative depression scales (see Table 3). Pre-surgical medications and doses were unchanged throughout the 6 month study.

TABLE 2
Hamilton Depression Rating Scale (HRDS-17)
scores over time for each subject

| | Hamilton Score | | | | | |
|---|---|---|---|---|---|---|
| Time | Pt 1 * | Pt 2 # | Pt 3 * | Pt 4 # | Pt 5 * | Pt 6 * |
| Pre-op Baseline | 29 | 22 | 29 | 24 | 26 | 25 |
| 1 week post-op | 5 | 10 | 12 | 18 | 17 | 12 |
| (acute stimulation) | | | | | | |
| 2 weeks post-op | 9 | 13 | 23 | 18 | 22 | n/a |
| (DBS off) | | | | | | |
| 1 month | 10 | 14 | 17 | 20 | 22 | 12 |
| 2 months | 13 | 11 | 12 | 18 | 10 | 12 |
| 3 months | 2 | 15 | 14 | 25 | 7 | 14 |
| 4 months | 4 | 9 | 12 | 24 | 6 | 12 |
| 5 months | 5 | 18 | 7 | 23 | 8 | n/a |
| 6 months | 5 | 15 | 9 | 23 | 6 | 12 |

Clinical response: decrease HDRS score >50%.
Clinical remission: absolute HDRS score <8

TABLE 3
Psychiatric Ratings: Patient Subgroups

| | All Patients (n = 6) | | | | Responders (n = 4) | | | | Non-Responders (n = 2) | | | |
|---|---|---|---|---|---|---|---|---|---|---|---|---|
| | mean scores (SD) | | | | mean scores (range) | | | | mean scores (range) | | | |
| | Pre-op | 1 mo | 3 mo | 6 mo | Pre-op | 1 mo | 3 mo | 6 mo | Pre-op | 1 mo | 3 mo | 6 mo |
| HDRS 17 | 25.8 | 15.8 | 12.8 | 11.5 | 27.3 | 15.3 | 9.3 | 7.8 | 23 | 17 | 20 | 19 |
| | (2.8) | (4.7) | (7.8) | (6.8) | (2.1) | (5.4) | (5.9) | (3.1) | (22-24) | (14-20) | (15-25) | (15-23) |
| HDRS 24 | 34.6 | 25.8 | 21.2 | 18.8 | 35.7 | 25 | 15.3 | 11.3 | 33 | 27 | 30 | 30 |
| | (1.9) | (9.1) | (8.7) | (10.6) | (1.5) | (12.5) | (3.5) | (2.9) | (32-34) | (24-30) | (27-33) | (27-33) |
| MADRS | 33.3 | 23 | 17.4 | 18.5 | 33.8 | 20.7 | 11 | 9.7 | 33 | 27 | 27 | 29 |
| | (4.5) | (8.5) | (10.1) | (10.4) | (6.7) | (11.1) | (3.6) | (3.8) | (31-34) | (25-28) | (21-33) | (25-33) |
| CGI | 6.2 | 5.2 | 4.2 | 4.0 | 6.3 | 4.7 | 3.3 | 3.0 | 6 | 6 | 6 | 6 |
| | (0.4) | (0.8) | (0.6) | (1.7) | (0.5) | (0.6) | (1.0) | (0.8) | (6) | (6) | (6) | (6) |

note:
f/u MADRAS and HDRS 24 scores not available for patient 6.

Normalization of early morning sleep disturbance (middle insomnia commonly seen in MDD) occurred in the first week of chronic DBS in 4 of the six subjects (patients 1, 3, 5, and 6) and was the first notable sustained symptom change. Over the initial few weeks of continuous DBS, increased energy, interest, and psychomotor speed were additionally reported, with effects generally appreciable a day or two following stimulation adjustments. Patients and their families described renewed interest and pleasure in social and family activities, decreased apathy and anhedonia, as well as an improved ability to plan, initiate and complete tasks that were reported as impossible to attempt prior to surgery. While all patients continued to report feeling ‘moderately depressed’ for several weeks, several also indicated that the sensations of ‘painful emptiness’ and ‘void’ remitted almost immediately following onset of stimulation at the optimal contacts.

Two patients failed to show a sustained antidepressant response at the six month time point (Table 2). One of these subjects, Patient #2, met criteria for clinical response in the first 4 months; however the level of improvement fluctuated over time and the maximal benefit could not be recaptured with either a change in the stimulation contact or adjustments in stimulation parameters after 4 months. Patient #4 had no appreciable clinical improvement with chronic stimulation despite trials with various combinations of contacts and stimulation parameters. Of note, the prominent sleep disturbances in these two patients (difficulty falling asleep (Patient #4) and hypersomnia (Patient #2)) were not affected by DBS, unlike the middle insomnia improvements seen in the other four subjects.

After a period of continuous stimulation for six months, the effects of cessation and re-introduction of stimulation was examined in subject #1 who had shown the earliest, most robust, and best sustained clinical response (Table 2). Following blinded discontinuation of bilateral stimulation (stimulators set at 0.0 V), antidepressant effects were maintained for two weeks (HDRS=9; PANAS positive score=48 of out of a possible 50, PANAS negative score=10 out of a possible 50 versus 6-month score Positive=50, Negative=10). In weeks 3 and 4 without stimulation, the improvements in mood were also sustained (HDRS=10). In the context of this sustained euthymia however, there was a progressive change in behavior characterized by loss of energy and initiative, impaired concentration, and reduced activities, reflected by a drop is the PANAS positive score to 37, without appreciable change in the negative score (negative=13). At this point, and under continued blinded conditions, the stimulator was turned back on to the previous best settings (3.5 V, PW 60, 130 Hz). This resulted in normalization of symptoms within approximately 48 hours and return to pre-discontinuation activities within one week (HDRS=6, PANAS positive=50, negative=10 after 1 week of restarting chronic DBS). This remission was sustained at 6 weeks of resumed stimulation at comparable levels to the pre-discontinuation baseline (HDRS=4). Taken together, these findings suggest that stimulation of Cg25WM produces long-term improvements in mood that are sustained beyond the period of active stimulation. The cognitive aspects of depression (i.e., poor concentration, apathy) also show sustained improvements, but the observed changes appear to have a different biology and kinetics, decaying closer to the cessation of stimulation.

### Example 4

### PET Scanning Acquisition and Data Analysis

Regional cerebral blood flow PET scans (rCBF) were acquired preoperatively and after 3 and 6 months of chronic DBS (Fox et al., 1984). Five CBF scans were acquired in each subject at each time point. A comparative scan-set (one time point only) was also acquired under identical scanning conditions in a group of 5 age- and sex-matched healthy volunteers. All scans were acquired with subjects resting, with eyes closed and no explicit cognitive or motor instructions. Scans were acquired on a GEMS/Scanditronix 2048b camera (15 parallel slices; 6.5 mm center-to-center inter-slice distance) using measured attenuation correction (68 Ge/68 Ga transmission scans). rCBF was measured using the bolus [15O]-water technique (35 mCi 15O-water dose/scan; scan duration 60 seconds) (Mayberg et al., 1999). Scans were spaced a minimum of 11 minutes apart to accommodate radioactive decay to background levels. Mood state (sadness and anxiety) was assessed at the end of each scan using a 7 point analogue scale and the PANAS to verify behavioral stability over the course of the 5 scans (Watson and Clark, 1988).

Positron emission tomography (PET) was used to characterize the activity in brain networks involved in TRD and to provide a quantitative measure of brain changes associated with stimulation. Baseline, resting-state, cerebral blood flow (CBF) PET scans were performed in the first 5 study subjects and compared to five age- and sex-matched, non-depressed healthy volunteers. Depressed patients showed a unique pattern of elevated subgenual cingulate (Cg25) blood flow at pretreatment baseline, not previously reported in studies of non-treatment resistant patients. In addition, and consistent with past studies of depressed patients (reviewed in Mayberg, 2003), CBF decreases in prefrontal (BA9/46), premotor (BA6), dorsal anterior cingulate (BA24), and anterior insula were also identified. (Table 4, left). A similar pattern of hyperactive Cg25 and hypoactive prefrontal cortex was seen in both the DBS responders and non-responders (data not shown). Responder versus non-responder differences at baseline were seen primarily in the magnitude of the prefrontal decreases (responders>non-responders). Responders also showed an area of hyperactivity in the medial frontal cortex (BA10) not seen in the non-responders, however the small sample size precluded further analysis.

Serial scans were performed after 3 and 6 months in 4 of the first 5 patients (#1, 2, 3 and 5). Group analyses showed local CBF decreases in Cg25 and adjacent orbital frontal cortex (BA11) after 3 months of stimulation. The long-term responders (Pts 1, 3, 5) showed additional CBF changes at both 3 and 6 months: decreases in hypothalamus, anterior insula, and medial frontal cortex (BA10) as well as increases in dorsolateral prefrontal (BA9/46), dorsal anterior (BA24) and posterior cingulate (BA31), premotor (BA6), and parietal (BA40) regions (Table 4). Neither the medial frontal (BA10) decreases, nor the dorsal prefrontal (BA9/46), anterior cingulate (BA24) or parietal (BA40) increases were seen in the non-responder (patient #2) at either 3 or 6 months.

The stimulation-induced CBF increases in prefrontal cortex (BA9/46) normalized pretreatment abnormalities. Similarly, the Cg25 decreases not only normalized pretreatment dysfunction, but activity in this region with DBS was actually suppressed below that of the controls at both time points, a change also observed in our previous studies of PET scans changes with response to antidepressant medications (Mayberg et al., 2000). Unlike medication, brainstem changes were not seen early with DBS, although changes in the pons were demonstrated at the 6 month time point. Overall, regional changes seen after 3 months were maintained at 6 months in all three responders (Table 4, middle and right sections).

TABLE 4
PET Blood Flow Changes

| Baseline¹ | | | | | | 3 months DBS vs Baseline²* | | | | | | 6 months DBS vs Baseline²* | | | | | |
|---|---|---|---|---|---|---|---|---|---|---|---|---|---|---|---|---|---|
| Patients (n = 5) vs Controls (n = 5)* | | | | | | Responders (n = 3) | | | | | | Responders (n = 3) | | | | | |
| | | | | z | x y z | | | | | z | x y z | | | | | z | |
| region | BA | | Δ | score | coordinates | region | BA | | Δ | score | coordinates | region | BA | | Δ | score | z coordinates |
| sgCg | 25 | L | ↑ | 5.16 | −10 28 −12 | sgCg | 25 | | ↓ | −4.75 | −2 8 −10 | sgCg | 25 | | ↓ | −3.88 | 10 20 −4 |
| Hth | | | ↓ | −3.67 | 0 4 −12 | | | | | | | Hth | | | ↓ | −4.63 | −2 2 −4 |
| | | | | | | OrbF | 32/10 | | ↓ | −5.1 | 0 34 −8 | OrbF | 32/11 | L | ↓ | −4.97 | −10 30 −10 |
| | | | | | | | 32/10 | R | ↓ | −4.84 | 6 46 2 | | | | | | |
| | | | | | | | 10 | R | ↓ | −3.98 | 22 60 −10 | | 11/10 | L | ↓ | −4.41 | −20 36 −10 |
| aIns | | L | ↑ | 6.05 | −38 22 14 | Ins | | R | ↓ | −3.76 | 60 10 −6 | Ins | | R | ↓ | −4.07 | 58 16 −6 |
| | | | | | | | | L | ↓ | −5.74 | −50 16 −16 | | | L | ↓ | −5.67 | −50 20 −6 |
| mFr | 10 | R | ↑ | 6.22 | 26 42 16 | mFr | 10 | L | ↓ | −4.19 | −22 62 22 | mFr | 10 | L | ↓ | −3.53 | −14 56 38 |
| | | | | | | | 10 | R | ↓ | −5.95 | 34 56 26 | | 10 | L | ↓ | −4.27 | −28 56 32 |
| | | | | | | | 24 | R | ↓ | −5.86 | 12 22 26 | | 24 | R | ↓ | −4.19 | 12 20 20 |
| | | | | | | | | | | | | | 24 | L | ↓ | −3.79 | −16 24 22 |
| | | | | | | mFr | 9 | R | ↓ | −5.57 | 12 46 34 | mFr | 9 | R | ↓ | −3.95 | 6 52 30 |
| | | | | | | | | L | ↓ | −4.87 | −5 56 34 | | 9 | R | ↓ | −3.34 | 2 46 40 |
| | | | | | | Fr pole | 9 | L | ↓ | −5.96 | −28 54 43 | | | | | | |
| | | | | | | | | R | ↓ | −5.19 | 18 52 34 | | | | | | |
| pCg | 31 | L | ↑ | 4.43 | −8 −54 26 | pCg | 31 | L | ↑ | 4.65 | −10 −72 20 | | | | | | |
| | | R | ↑ | 3.88 | 10 −56 26 | | 31 | R | ↑ | 3.51 | 24 −68 14 | | | | | | |
| DLPF | 9 | L | ↓ | −7.07 | −38 28 30 | DLPF | 9 | L | ↑ | 4.05 | −26 16 30 | DLPF | 9 | R | ↑ | 4.63 | 38 12 34 |
| | | R | ↓ | −4.92 | 40 20 30 | | | | | | | | 46/9 | L | ↑ | 3.79 | −34 18 24 |
| VLPF | 47 | L | ↓ | −4.66 | −52 42 2 | | 46 | L | ↑ | 4.73 | −38 32 18 | | 46 | R | ↑ | 4.13 | 32 24 10 |
| | | R | ↓ | −4.98 | 32 26 0 | | 46 | R | ↑ | 4.91 | 34 22 16 | | 46 | L | ↑ | 3.84 | −38 26 10 |
| dCg | 24b | L | ↓ | −5.39 | −2 18 28 | dCg | 24b | L | ↑ | 4.06 | −2 10 28 | dCg | 24b | L | ↑ | 3.58 | −4 4 34 |
| | | | | | | PM | 6 | L | ↑ | 4.21 | −52 −4 32 | PM | 6 | L | ↑ | 3.34 | −48 2 22 |
| | | | | | | | 6 | R | ↑ | 5.71 | 50 0 30 | | 6 | R | ↑ | 5.92 | 50 4 28 |
| | | | | | | | | | | | | Par | 39 | L | ↑ | 4.13 | −36 −56 18 |
| | | | | | | | | | | | | | 39 | R | ↑ | 4.01 | 44 −56 10 |
| | | | | | | | | | | | | PAG | | R | ↓ | −4.66 | 6 −36 −10 |
| Caudate | | R | ↓ | −4.69 | 14 2 12 | | | | | | | | | | | | |
| aThal | | R | ↓ | −4.58 | 12 −6 14 | | | | | | | | | | | | |
| n. acc | | L | ↓ | −4.68 | −4 10 −4 | | | | | | | | | | | | |

¹pts 1-5 vs 5 age and gender matched healthy controls.
²pts 1, 3 and 5.
*SPM t-maps: significance threshold: p < .001 uncorrected (t > 3.27); cluster size >50 voxels.

### Example 5

### Neuropsychological Testing

A comprehensive battery of neuropsychological tests was administered at three time points to establish baseline intellectual and cognitive abilities prior to surgery/stimulation, and to monitor for changes over time (3 months, 6 months). Tests were chosen to tap general cognitive and intellectual function, as well as four domains of frontal function (Bechara et al. 1994; Freedman et al., 1998; Lang et al., 1999; Spreen and Strauss, 1998). Parallel versions were used where possible to minimize effects of repetition, and scores are corrected for effects of age, gender and education, where appropriate. The following tests were administered: Wechsler Adult Intelligence Scale-III; North American Adult Reading Test; Trail Making Tests A and B; Boston Naming Test; Benton Judgment of Line Orientation Test; Hopkins Verbal Learning Test; Brief Visual Spatial Memory Test, Revised; Finger Tapping Test; Grooved Pegboard Test; Controlled Oral Word Association Test; Wisconsin Cart Sorting Test; Stroop Color Word Test; Emotional Stroop Task; Object Alternation Test; Iowa Gambling Task, and the International Affective Picture System Ratings. A sub-set of tests is presented. Paired t-tests were used to compare differences between the baseline and six months data to determine the probability that the actual mean difference is consistent with zero. This comparison is aided by the reduction in variance achieved by taking the differences, and thus is a good choice for use with a small sample.

At baseline, all patients were functioning intellectually in the average range, consistent with estimates of premorbid IQ. Inspection of results over time indicates that surgery itself did not have a negative impact on general cognition (i.e., IQ, language, basic visual-spatial function). Moreover, many specific areas that were below average or impaired at baseline were significantly improved (or trending due to low power) following 6 months of DBS (responders: visuo-motor function, particularly with the non-dominant hand, t(2)=5.8, p=0.014; dorsolateral frontal function (verbal fluency), t(2)=10.0, p=0.005; ventral prefrontal function (fewer errors on object alternation task), t(2)=1.7, p=0.12; and orbital frontal function (fewer risky choices on the gambling task), t(2)=6.3, p=0.012). Importantly, there were no acquired impairments in orbital frontal functioning to indicate local DBS adverse effects (Kartsounis et al., 1991; Dalgleish et al., 2004). Non-responders had normal performance on all tests at baseline, with the exception of slowed psychomotor speed (consistent with effects of depression). Repeat testing was only available for one of these subjects.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
A.P.A. (2000). Practice guideline for the treatment of patients with major depressive disorder (revision). Am J Psychiatr. 157, 1-45.
Barbas, H., Saha, S., Rempel-Clower, N. & Ghashghaei, T. (2003). Serial pathways from primate prefrontal cortex to autonomic areas may influence emotional expression. BMC Neurosci. 4, 25.
Bechara, A., Damasio, A. R., Damasio, H. & Anderson, S. W. (1994). Insensitivity to future consequences following damage to human prefrontal cortex. Cognition 50: 7-15.
Benabid, A. L. (2003). Deep brain stimulation for Parkinson's disease. Curr Opin Neurobiol. 13, 696-706.
Brody, A. L., Barsom, M. W., Bota, R. G., Saxena, S. (2001). Prefrontal-subcortical and limbic circuit mediation of major depressive disorder. Semin Clin Neuropsychiatry. 6, 102-12.
Carmichael, S. T. & Price, J. L. (1996). Connectional networks within the orbital and medial prefrontal cortex of macaque monkeys. J Comp Neurol. 371, 179-207.
Caspi, A., Sugden, K., Moffitt, T. E., Taylor, A., Craig, I. W., Harrington, H., McClay, J., Mill, J., Martin, J., Braithwaite, A., Poulton, R. (2003). Influence of life stress on depression: moderation by a polymorphism in the 5-HTT gene. Science 301, 386-9.
Dalgleish, T., Yiend, J., Bramham, J., Teasdale, J. D., Ogilvie, A. D., Malhi, G., Howard, R. (2004). Neuropsychological processing associated with recovery from depression after stereotactic subcaudate tractotomy. Am J Psychiatry. 161, 1913-6.
Davis, K. D., Taub, E., Houle, S., Lang, A. E., Dostrovsky, J. O., Tasker, R. R., Lozano, A. M. (1997). Globus pallidus stimulation activates the cortical motor system during alleviation of parkinsonian symptoms. Nat Med. 3, 671-674.
Deep-Brain Stimulation for Parkinson's Disease Study Group (2001). Deep-brain stimulation of the subthalamic nucleus or the pars intema of the globus pallidus in Parkinson's disease. N Engl J Med. 345, 956-63
Dougherty, D. D., Weiss, A. P., Cosgrove, R., Alpert, N. M., Cassem, E. H., Nierenberg, A. A., Price, B. H., Mayberg, H. S., Fischman, A. J., Rauch, S. L. (2003). Cerebral Metabolic Correlates as Potential Predictors of Response to Cingulotomy for Major Depression. J Neurosurg, 1010-1017.
Drevets, W. C., Price J. L., Simpson, J. R. Jr, Todd, R. D., Reich, T., Vannier, M., Raichle, M. E. (1997): Subgenual prefrontal cortex abnormalities in mood disorders. Nature 386, 824-827
Drevets, W. C., (1999). Prefrontal cortical-amygdalar metabolism in major depression. Ann N Y Acad Sci. 877, 614-37.
Fava, M. (2003). Diagnosis and definition of treatment-resistant depression. Biol Psychiatry 53, 649-659.
First, M. B., Spitzer, R. L., Gibbon, M., Williams, J. B. W. (2001). Clinical Interview for DSM-IVTR (SCID-I): User's Guide and Interview-Research Version. New York, New York Psychiatric Institute, Biometrics Research Department.
Fox, P. T., Mintun, M. A., Raichle, M. E. & Herscovitch, P. (1984). A noninvasive approach to quantitative functional brain mapping with H2 (15)O and positron emission tomography. J Cereb Blood Flow Metab. 4, 329-33.
Frank, E., Prien, R. F., Jarrett, R. B., Keller, M. B., Kupfer, D. J., Lavori, P. W., Rush, A. J., Weissman, M. M. (1991). Conceptualization and rationale for consensus definitions of terms in major depressive disorder. Remission, recovery, relapse, and recurrence. Arch Gen Psychiatry. 48:851-5
Freedman, L. J., Insel, T. R. & Smith, Y. (2000). Subcortical projections of area 25 (subgenual cortex) of the macaque monkey. J Comp Neurol 421, 172-88.
Freedman, M., Black, S., Ebert, P. & Binns, M. (1998). Orbitofrontal function, object alternation and perseveration. Cerebral Cortex, 8, 18-27.
Gabbott, P. L., Jays, P. R. & Bacon, S. J. (1997). Calretinin neurons in human medial prefrontal cortex (areas 24a,b,c, 32′, and 25). J Comp Neurol. 381, 389-410.
Goldapple, K., Segal, Z., Garson, C., Lau, M., Bieling, P., Kennedy, S., Mayberg, H. (2004). Modulation of cortical-limbic pathways in major depression: treatment specific effects of cognitive behavioral therapy. Arch Gen Psych 61:34-41.
Haber, S. N. (2003). The primate basal ganglia: parallel and integrative networks. J Chem Neuroanatomy 26, 317-330.
Hamilton, M. A. (1960). Rating Scale for Depression. J Neurol Neurosurg Psychiatry 23, 56-62.
Heim, C., Newport, D. J., Heit, S., Graham, Y. P., Wilcox, M., Bonsall, R., Miller, A. H., Nemeroff, C. B. (2000). Pituitary-adrenal and autonomic responses to stress in women after sexual and physical abuse in childhood. JAMA 284, 592-597.
Hodaie, M., Wennberg, R. A., Dostrovsky, J. O., Lozano, A. M. (2002). Chronic anterior thalamus stimulation for intractable epilepsy. Epilepsia. 43, 603-8.
Hyman, S. E. & Nestler, E. J. (1996). Initiation and Adaptation: a paradigm for understanding psychotropic drug action. Am J Psychiatry. 153, 151-162.
Jurgens, U. & Muller-Preuss, P. (1977). Convergent projections of different limbic vocalization areas in the squirrel monkey. Exp Brain Res 29, 75-83.
Kartsounis, L. D., Poynton, A., Bridges, P. K. & Bartlett, J. R. (1991). Neuropsychological correlates of stereotactic subcaudate tractotomy. A prospective study. Brain 114, 2657-73.
Keller, M. B., Lavori, P. W., Mueller, T. I., Endicott, J., Coryell, W., Hirschfeld, R. M., Shea, T (1992). Time to recovery, chronicity, and levels of psychopathology in major depression. A 5 year prospective followup of 431 subjects. Arch. Gen. Psychiatry 49, 809-816.
Kendler, K. S., Thornton, L. M. & Gardner, C. O. (2001). Genetic Risk, number of previous depressive episodes, and stressful life events in predicting onset of major depression Am J Psychiatry. 158, 582-586.
Kennedy, S. H & Lam, R. W (2003). Enhancing outcomes in the management of treatment resistant depression: a focus on atypical antipsychotics Bipolar Disord. 5 Suppl 2, 36-47.
Kumar, R., Lozano, A. M., Sime, E., Lang, A. E. (2003). Long-term follow-up of thalamic deep brain stimulation for essential and parkinsonian tremor. Neurology 9, 61, 1601-4.
Lancet UK ECT Review Group (2003). Efficacy and safety of electroconvulsive therapy in depressive disorders: a systematic review and meta-analysis. Lancet, 361, 799-808.
Lang, A. E. & Lozano, A. M. (1998). Parkinson's disease. Second of two parts. N Engl J Med. 339, 1130-1143.
Lang, P. J., Bradley, M. M. & Cuthbert, B. N. (1999). International affective picture rating system (IAPS): Instruction manual and affective ratings. Technical Report A-4, The Centre for Research in Psychophysiology, University of Florida.
Lozano, A. M., Dostrovsky, J., Chen, R. & Ashby, P. (2002). Deep brain stimulation for Parkinson's disease: disrupting the disruption. Lancet Neurol. 1,:225-31.
Maclean, P. D. (1990). The Triune Brain in Evolution: Role in Paleocerebral Function, New York, N.Y., Plenum.
Malizia, A. (1997). Frontal lobes and neurosurgery for psychiatric disorders. J Psychopharm, 11, 179-187.
Manji, J. K., Drevets, W C., Charney, D. S. (2001). The cellular neurobiology of depression. Nature Medicine 7:541-547.
Mayberg, H. S. (1994). Frontal lobe dysfunction in secondary depression. J. Neuropsych. Clin. Neurosci., 6, 428-442.
Mayberg, H. S. (1997). Limbic-cortical dysregulation: a proposed model of depression. J Neuropsychiatry Clin Neurosci 9, 471-481.
Mayberg, H. S. (2003). Modulating Dysfunctional Limbic-Cortical Circuits in Depression: Towards Development of Brain-based Algorithms for Diagnosis and Optimised Treatment. Br Med Bull. 65, 193-207.
Mayberg, H. S., Brannan, S. K., Mahurin, R. K., McGinnis, S., Silva, J. A., Tekell, J. L., Jerabek, P. A., Martin, C. C., Fox, P. T. (2000). Regional metabolic Effects of fluoxetine in major depression: serial changes and relationship to clinical response. Biol Psychiatry, 48, 830-843.
Mayberg, H. S., Liotti, M., Brannan, S. K., McGinnis, S., Mahurin, R. K., Jerabek, P. A., Silva, J. A., Tekell, J. L., Martin, C. C., Fox, P. T. (1999). Reciprocal limbic-cortical function and negative mood: converging PET findings in depression and normal sadness, Am J Psychiatry, 156, 675-682.
McIntyre, C. C., Savasta, M., Kerkerian-Le Goff, L. & Vitek, J. L. (2004). Uncovering the mechanism(s) of action of deep brain stimulation: activation, inhibition, or both. Clin Neurophysiol. 115, 1239-48.
Mottaghy, F. M., Keller, C. E., Gangitano, M., Ly, J., Thall, M., Parker, J. A., Pascual-Leone, A. (2002). Correlation of cerebral blood flow and treatment effects of repetitive transcranial magnetic stimulation in depressed patients. Psychiatry Research Neuroimaging 115, 1-14
Montgomery, S. A. & Asberg, M. (1979). A new depression scale designed to be sensitive to change. Br J Psychiatry. 134, 382-9.
National Institute of Mental Health (1970). CGI: Clinical Global Impressions. In: Guy, W., Bonato, R. R., eds. Manual for the ECDEU Assessment Battery. 2. (Chevy Chase, M D: National Institute of Mental Health), 12-1-12-6.
Nestler, E. J., Barrot, M., DiLeone, R. J., Eisch, A. J., Gold, S. J., Monteggia, L. M. (2002). Neurobiology of Depression. Neuron 34:13-25.
Nemeroff, C. B (2002). Recent advances in the neurobiology of depression. Psychopharmacol Bull 36 Suppl 2, 6-23.
Nobler, M. S., Oquendo, M. A., Kegeles, L. S., Malone, K. M., Campbell, C. C., Sackeim, H. A., Mann, J. J. (2001) Decreased regional brain metabolism after ECT. Am J Psychiatry, 158, 305-8.
Ongur, D., An, X. & Price, J. L. (1998). Prefrontal cortical projections to the hypothalamus in Macaque Monkeys. J Comp Neurol 401, 480-505.
Sackeim, H. A., Rush, A. J., George, M. S., Marangell, L. B., Husain, M. M., Nahas, Z., Johnson, C. R., Seidman, S., Giller, C., Haines, S., Simpson, R. K. Jr, Goodman, R. R. (2001). Vagus nerve stimulation (VNS) for treatment-resistant depression: efficacy, side effects, and predictors of outcome. Neuropsychopharmacology. 25, 713-28.
Schaltenbrand, G. & Wahren, W. (1977). Atlas for stereotaxy of the human brain, 2nd ed. (Stuttgart, Germany: Thieme Verlag).
Seminowicz, D. A., Mayberg, H. S., McIntosh, A. R., Goldapple, K. K., Kennedy, S., Segal, Z., Rafi-Tari, S. (2004). Limbic-Frontal Circuitry in Major Depression: A Path Modeling Metanalysis. Neuroimage, 22, 409-418.
Spreen, O. & Strauss, E. A. (1998). Compendium of Neuropsychological Tests, 2nd ed. (New York, N.Y.: Oxford University Press).
Thase, M. E. & Rush, A. J. (1997). When at first you don't succeed: Seuqntial strategies for antidepressant nonrsponders. J. Clin. Psychiatry. 58, Suppl 13, 23-29.
Vaidya, V. A., Duman, R. S. (2001). Depresssion-emerging insights from neurobiology. Br Med Bull. 57:61-79.
Vogt, B. A. & Pandya, D. N. (1987). Cingulate cortex of the rhesus monkey II: Cortical afferents. J Comp Neurol. 262, 271-289.
Wang, P. S. (2003). National Co-morbidity survey replication. The epidemiology of major depressive disorder: results from the National co-morbidity Survey Replication (NCS-R). JAMA. 289, 3095-3105.
Watson, D. & Clark, L. A. (1988). Development and Validation of Brief Measures of Positive and Negative affect: the PANAS scales. Personality Soc Psychol 6, 1063-1070.
World Health Organization (2001). Chapter 2: Burden of Mental and Behavioural Disorders in The WHO Report 2001: Mental Health, New Understanding, New Hope http://www.who.int/whr/2001/chapter2/en/index3.html.
Worsley K J, Marrett S, Neelin P, Vandal A C, Friston K J, Evans A C. (1996): A unified statistical approach for determining significant signals in images of cerebral activation. Hum Brain Mapping 4, 58-73.
U.S. Pat. No. 3,731,681
U.S. Pat. No. 3,951,147
U.S. Pat. No. 4,692,147
U.S. Pat. No. 4,772,263
U.S. Pat. No. 5,119,832
U.S. Pat. No. 5,263,480
U.S. Pat. No. 5,299,569
U.S. Pat. No. 5,423,877
U.S. Pat. No. 5,458,631
U.S. Pat. No. 5,470,846
U.S. Pat. No. 5,540,734
U.S. Pat. No. 5,735,505
U.S. Pat. No. 5,840,069
U.S. Pat. No. 6,016,449
U.S. Pat. No. 6,036,459
U.S. Pat. No. 6,051,017
U.S. Pat. No. 6,176,242
U.S. Pat. No. 6,251,669
U.S. Pat. No. 6,425,852
U.S. Pat. No. 6,609,031
U.S. Pat. No. 6,620,151
U.S. Pat. No. 6,666,845
U.S. Pat. No. 6,735,474
U.S. Pat. No. 6,735,475
U.S. Publication No. US20040092010

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

We claim:

1. A method of treating a cognitive impairment and/or disorder comprising the steps of:
surgically implanting an electrode in communication with a predetermined area;
coupling the electrode to a pulse generating source; and
generating an electrical signal with the pulse generating source wherein said signal electrically stimulates the area thereby treating the cognitive impairment and/or disorder.

2. The method of claim 1 further comprising the steps of:
surgically implanting a catheter having a proximal end coupled to a pump and a discharge portion for infusing a dosage of a pharmaceutical, wherein after implantation the discharge portion of the catheter is in communication with the area; and
operating the pump to discharge the pharmaceutical through the discharge portion of the catheter into the area thereby treating the cognitive impairment and/or disorder.

3. The method of claim 1, wherein the predetermined site is selected from the group consisting of subcallosal area, subsingular cingulate, orbital frontal cortex, anterior insula, medial frontal cortex, dorsolateral prefrontal area, posterior cingulate area, premotor cortex, parietal region, ventrolateral prefrontal cortex, caudate, anterior thalamus, nucleus accumbens; periaqueductal gray area, and brainstem.

4. The method of claim 1, wherein the cognitive disorder is selected from the group consisting of learning acquisition, memory consolidation, and retrieval.

5. The method of claim 1, wherein the cognitive disorder is associated with a disorder from the group consisting of Alzheimer's disease, Parkinson's disease, Creutzfeld-Jacob disease, attention deficit hyperactivity disorder, dementia and stroke.

6. The method of claim 2, wherein the pharmaceutical is selected from the group consisting of an inhibitory neurotransmitter agonist, an excitatory neurotransmitter antagonist, an agent that increases the level of an inhibitory neurotransmitter, an agent that decrease the level of an excitatory neurotransmitter, and a local anesthetic agent.

7. A method of treating a cognitive impairment and/or disorder comprising the steps of:
surgically implanting an electrode in communication with a predetermined area;
surgically implanting a catheter having a proximal end coupled to a pump and a discharge portion for infusing a dosage of a pharmaceutical, wherein after implantation the discharge portion of the catheter is in communication with the area; and
coupling the proximal end of the lead to a pulse generating source;
generating an electrical signal with the pulse generating source wherein said signal electrically stimulates the area; and
operating the pump to discharge the pharmaceutical through the discharge portion of the catheter into the area thereby treating the cognitive impairment and/or disorder.
